SEQUENCE LISTING (128 sequences)

SEQ ID NO: 1 - protein, 785 aa, Canis lupus
MSRVLWTLWVLGAVTNLSKEEAPDQSSSLSCDPTGVCDGRSRSLNSMPSGLTAAVRSLDLSNNEITYIGNSDLRDCVNLKALRLESNGINTIEEESFFSLWSLEHLDLSYNLLSNLSSSWFRPLSSLKFLNLLGNPYKSLGETPLFSQLTNLRILKVGNIYSFTEIQDKDFAGLTFLEELEIDASNLQRYEPKSLKSIQSISYLALRMKQPVLLVEIFVDLSSSLKHLELRDTHLDTFHFSEASINETHTLVKKWTFRNVKVTDRSFTEVVRLLNYVSGVLEVEFEDCTLYGLGDFDIPDVDKIKNIGQIETLTVRRLHIPHFYSFYDMSSIYSLTEDVKRITVESSKVFLVPCSLSQHLKSLEYLDLSDNLVVEEYLRNSACQHAWPLLQTLILRQNRLKSLEKTGETLLTLKNLVNLDISKNNYLSMPETCQWPEKLKCLNLSDTRMQSITRCIPQTLEILDVSNNNLESFSLILPQLKELSISRNKLKTLPDASFLPTLQIMRISRNTINAFSKEQLDSFHRLQTLEAGGNNFLCSCEFLSFTQEQQALAGLLVGWPEDYLCHSPSYVRGQRVGTARLPASECHRTALVAAVCCVLLLLVLLTAGACHHFHGLWYLRMLWAWLQAKRKPRKAPSRDVCYDAFVSYSEHDSYWVENLLVQKLEHFNPPFKLCLHKRDFIPGKWIIDNIIDSIEKSHKTIFVLSENFVKSEWCKYELDFSHFRLFDENNDAAILILLEPIEKKAIPQRFCKLRKIMNTKTYLEWPTDDAQQEGFWLNLRTAIKS

SEQ ID NO: 2 - protein, 1422 aa, Canis lupus
MGGERACRGSSTCQVPALEVRGEAAGAPVLHPPPPRWASFLCIPGAPGCHRVIGGLPGHSGRCRLPQDVRPSWPVCPAPAAPCAQSGRVALVQMTGALSRAALAPTGLGRGWEGDALTRQAGRFADGGPKDQRVRADPGRVWSAQGAEGRAPGTGRGRHQYPPPAIFPEALHASRLSDGKQTLLKVSVSQGPSTALSARPAGRTCGGRQVPVQGPPSWAPGWRWACTAVFPSRRIMGRQLGRTLGLLLVAGAVAAASCCVADGRRALYRSCNLSQVPPVPSTTEILLLSFNYIRAVTRASFPLLERLQLLELGTQQTPFSVDREAFRNLPNLRTLDLGNSRVDFLHPDAFQGLPHLQELRLFACGLSDVVLTDGYFRNLGALLRLDLSKNQIGSLELHASFRELGSLRSVDFSLNRIPAACEQGLRPLQGKALSLLNLAANGLYSRAPVDWGRCGNPFRNVVLETLDVSNNGWTADVTGNVTRAIGGSQISSLVLAHHIMGQGFGFRNIRDPDRSTFAGLAGSSVLRLDLSHGFVFSLNARLFEVLGDLKLLDLAHNKINRIAGEAFHGLGSVQVLNLSHNLLGELYDSDFSGLAEVAYIDLQHNHIGIIQDQTFRFLGALRTLDLRDNALKTVSFVPSIDTIFLGNNKLETVSHMDLTASFLELSDNRLEDLGDLYSLLRVPALQVLILNRNRLSACRGGHGPTGSVGPERLFLGSNMLQLAWETGRCWDVFRGLPRLRVLHLNHNYLAALPPGLLRDLTALRGLDLSANRLSTLSRGDLPAALEVLDVSRNQLLSLDPGLLAPLRAVDLTHNKFICGCELRPLVRWLNRTNVTVFGSRADVRCAYPSLLAGTPLSSVSMEGCDDEEALRTLTFSLFIFSTVGVTLFLLAVLVAAKLRGLCFLCYKAARRLLPAGPAEDGAPDAYQYDAYLCFSGRDFEWVQRALLRHLDAQYSSRNRLNLCFEERDFVPGREHIANIQDAVWSSRKVVCLVSRHFLRDGWCLEAFAAARSRCASHLDGALVLVVVGSLSQYQLRRHPAIGGFVRQRRYLRWPEDLQDVGWFLDTLSRHILQEQRGARGDGGIPLRTVAAGRRPHCTRVGRRRPHCTRVRGRRPHCTGAPGGADLTAPGSGGRRPHCTGVRGTPTSLHRGPGGRRPHCTGAPGGADLTAPGSGGADLSAPGTGGADLTAPGPRGRCSSAAGARVRQRARARAVRRRSSGSARRTREHTRPEEPPRARPALGRAPAAAPAPRAPARPRRRPPRLPRPCPPSARAGCPAPRPYPVSRPRGAGGGGGGGGGGGAGTRPRRRQAPPTRRPRCGPGAGFRLPPEGVPSRTGSPAPAAEGGGRGRSCQQFGGAGGHTDVRGLPPASGGPRVSPSARSSPDRRVAAGEGAGRGGTQEGRGVGEAGAGRERGGAGRGVG

SEQ ID NO: 3 - protein, 780 aa, Canis lupus
MIKDKDSITGSFHFVYIVTLIVGTIIQFSDESEFTVDMSNMNLTHVPEDLPPKTKILDMSQNNISELHLSDMSYLSGLKILRISHNRIWWLDFSIFKFNQDLEYLDLSYNQLRNMSCHLIRSLKHLDLSFNDFHVLPICKEFGNLTQLQFLGLSATKLRQLDLLPIAHLHLSYILLDLQGYYAKESEKGSLQILDTKTLHLVFHPNQLFSVQANMLVNNLGCLQLTNIKLNNDNCQVLIQFLSELTRGPTLLNFTLQHVKTTWKCLVRIFKFLWPKPVQYLNIYNLTIVESINKEYIHYPKTALKALTIEHVKNEVFLFSQTALYTIFSEMNIMMLTISDTPFIHMLCPPPSNTFKFLNFTQNVFTDSVFQSCSHLVRLETLILRKNKLKDLYKVGLMTKHMTSLEILDVSVNSLEYDRYDGNCTWVGSIVVLNLSSNILTDSVFRCLPPKVKVLDLHDNRIRSIPKPIMKLEDLQELNVASNSLAHFPDCGTFNRLSVLIIDSNSISNPSADFLQSCHNIRSMSAGNNPFQCTCELREFVQSLGQVASKVVEGWPDSYKCDSPENYKGTLLKDFHVSPLSCNTTLLLVTIGVAVLVFTVTVTALCIYFDLPWYLRMVFQWTQTRRRARNTPLEELQRTIQFHAFISYSEHDSAWVKNELVPCLEKEELRICLHERNFIPGKSIVENIINCIEKSYKSIFVLSPNFVQSEWCHYELYFAHHNLFHEGSNNLILILLEPIPQNCIPSKYHKLRALMTQRTYLEWPKEKSKHGLFWANIRAA

SEQ ID NO: 4 - protein, 1038 aa, Canis lupus
MSPRSLVLTCLFLLISDSYEFVTKANYSRSYPCDERRQNGSVIAECNGRRLQEVPQTVGKYVTVLDLSDNYITHITNESFHGLQNLTKINLNHNANPQHLSENPDNKNGMNITDGAFLNLQNLNQLLLEDNQLYQIPAGLPGSLKELSLIQNNIIWVTKKNTSGLTNLERLYLSWNCYFGNNCNNKTFNIEDGTFESLTNLEVLSLSFNKLVHVPPKLPSSLKELYLSNAKIKIISQEDFKGLRNLRVLDLSGNCPRCFNAPFPCTPCEGGASIQIHPLAFQTLTELRYLNLSSTSLRKIPATWFDNMRNLKVLHLEFNYLVDEIASGEFLTKLPVLEILDLSYNYVKAKYPKYINISHNFSSLKLLQALHLRGYVFQELRAGDFEPLMGLSNLKTINLGVNFIKQINFTLFQNFPNLSIIYLSENRISPLVNDIRQNEVNGSSSQRHVLKPRSADMEFDPHSNFYHNTHPLIKPQCTVYGKALDLSLNSIFFIGREQFEAFHDIACLNLSSNGNGQVLHGNEFSAVPHIKYLDLTNNRLDFDDDNALSDLPELEVLDLSYNAHYFRIAGVTHRLGFIQNLTQLKVLNLSHNSIYTLTEQDLRSVSLEELVFSGNRLDILWNAEGDKYWKIFTRLRNLTRLDLSLNNLRRIPNEAFLNLPQSLTQLYIKNNALNFFNWTLLQEFPRLQVLDLSGNRLSSITNSLSKFTSSLQTLLLHRNRISHLPASFLSEASSLIHLDLSSNLLKMINKSTLQTKTNTSLAILELGRNPFDCTCDIGDFRRWMDENLNVTIPRLTDVICSSPGDQRGKSIVSLELTTCISDTLAAVLCIFTSFITVTVMLAALGHHWFYWDVWFIYHVCLAKVKGYRSVSTSQTFYDAYVSYDTKDASVTDWVINELRFHLEESEGKNVLLCLEERDWDPGLAIIDNLMQSINQSKKTIFVLTKEYAQNWNFKTAFYLALQRLMDENMDVIIFILLEPVLQHSQYLRLRQRICKSSILQWPDNPKAEGLFWQSLKNVVLTENDSRYNNLYVDSIKQY

SEQ ID NO: 5 - protein, 790 aa, Canis lupus
MMKTNPSIFQFAIIFILILEIRIQLSEESDFLVNRSKAGLFHIPKDLSLKTTILDISQNYISELQTSDILSLSKLRILIVSYNRIQYLDISVFKFNQELEYLDLSHNELGRISCHPTVNLKHLDLSFNAFDDLPICKEFGNMSQLEFLGLSATQLQKSSMLPIASLHIRKVLLVLGDTYGKKEDPESLQKLNTESLHIVFPIRKEFSFTLDVSVSTAVSLELSNIKCVPDGHGWSYFQNVLSKLQKNSRLSSLTLNNIETTWNFFIMLLQLVWHTSIEYFSISNVKLQGYPDFRDFDYSDTSLKALSIHQVVSNAFNLPQSYIYKIFSNMNIQNFTVSGTHMVHMVCPSQISPFLHLDFSNNLLTDIVFKNCRNLIKLETLSLQMNQLKELASIAQMTNEMKSLQQLDISQNSLRYDENEGNCSWTRSLLSLNMSSNILTDSVFRCLPPKVKVLDLHDNRIRSIPKPIMKLEDLQELNVASNSLAHFPDCGTFNRLSVLIIDSNSISNPSADFLQSCHNIRSISAGNNPFQCTCELREFVQSLGQVASKVVEGWPDSYKCDSPENYKGTLLKDFHVSPLSCNTTLLLVTIGVAVLVFTVTVTALCIYFDLPWYLRMVFQWTQTRRRARNTPLENLQRTIQFHAFISYSGHDSAWVKSELLPNLEKEELRICLHERNFIPGKSIVENIINCIEKSYKSIFVLSPNFVQSEWCHYELYFAHHNLFHEGSNNLILILLEPIPQYSIPSSYHKLKNLMAQRTYLEWPKEKSKHGLFWANLRASINIKLREQAKK

SEQ ID NO: 6 - protein, 905 aa, Canis lupus
MSQSLLYHIYSFLGLLPFWILCTSSTNKCVVRHEVADCSHLKLTQVPDDLPANITVLNLTHNQLRRLPPANFTRYSQLTILDGGFNSISKLEPELCQKLPLLEILNLQHNELSHLSDQTFVFCVNLTELHLMSNSIKIIQNNPFRSLKNLVKLDLSHNGLSSTKLGSQLQLENLQELLLSNNKINVLRREELDFLGNSSLEKLELSSNPIKEFSPGCFHAIGKLFGLSLNNVQLNPSLTENLCLELSNTSIQNLSLSNTQLHRTSNMTFLGLKHTNLTMLDLSHNNLNVIENNSFVWLPHLEYFLLEYNNIEHLFSHSFYGLLNVRYLDLKRSFAKQSTSLASHPRIDDFSFQWLKCLQYLNMEDNYFAGIKSNMFTGLIKLKHLSLSNSFTSLQTLTNETFLSLAQSPLITLNLTKNKISKIESGAFSWLGHLQVLDLGLNEIGQELTGQEWRGLENIVEIYLSYNKYLQLTSSSFALIPSLRRLMLRRTALRNVDSSPSPFHPLRNLNILDLSNNNIANINDELLEGLEKLEILDMQHNNLARLWKHANPGGPVHFLKGLSHLHILNLESNGFDEIPAEVFKGLSELKSIDLGLNNLNIFPSSLFNDQVSLKSLNLQKNLITSVEKNVFGPAFRNLSNLDMSFNPFDCTCESIAWFVNWINSTHTNISELSSHYLCNTPPQYHGFPVMLFDISPCKDSAPFEIFFIINTSVLLTFIFIVLLIHFEGWRISFYWNVSVHRILGFKEIDKQPEQFEYAAYIIHAYKDRDWVWEHFSPMEEKDETLKFCLEERDFEAGVLELESIINSIKKSRKTIFVITQHLLKDPLCKRFKVHQAVQQAIEQNLESIILIFLEEIPDYKLNHALCLRRGMFKSHCILNWPVQKERVNAFHHKLQVALGSRNSIH

SEQ ID NO: 7 - protein, 833 aa, Canis lupus
MMSPTRLVGILIPAMAFLSCLRPESWDPCMQVVANTTYQCMELNLSKIPNNIPTSTEKLDLSFNPLRHLGSHCFSNFPKLQVLDLSRCEIQVIEDDAYQGLNHLSILILTGNPIQRLFPRAFSGLSSLKTLVAKETKLTSLEDFPIGHLKTLKELNVAHNLIHSFKLPAYFSNMPNLENVDLSNNKIQNIYREDLQVLHHMPLLNLSLDLSLNPLYFIQPGSFKEIKLHKLTLRSNFNSTDVMKTFIQGLAGLKINQLVLGEFKNERKLESFDNSLLEGLCNLTIEKFRIAYFDSFSKDTTNLFNQLVNISAISLAHLYLDTPKYLPKNLRWQRLEIVNCNLEQFPAWELDSLKEFVLTSNKGMNTFADMKMESLEFLDLSRNRLSFKTCCSHSDFGTTRLKHLDLSFNEIITMSSNFLGLEQLEYLDLQHSSLKQASDFSVFLSLRNLRYLDISYTRTEVAFQGIFDGLVSLEVLKMADNSFPDNSLPNIFKGLTNLTILDLSRCHLERVSQESFVSLPKLQEINMSHNSLLSLDTLAYEPLLSLQILDCSFNRIVAFKEQGQQHFPSNLVSLNLTRNNFACDCEHQSFLQWVKDHRQLLVEVEQMVCAKPLDMKDMPLLSFRNATCQRSKTIISVSVFTVLMVSLVAVLAYKFYFHLMLLAGCKRYNRGESTYDAFVIYSSQDEDWVRNELVKNLEEGVPPFQLCLHYRDFIPGVAIAANIIQEGFYKSRKVIVVVSQHFIQSRWCIFEYEIAQTWQFLSSRAGIIFIVLQKVEKSLLRQQVELYRLLSRNTYLEWEDSVLGRHIFWRRLRKALLDGKPWSPEGTEDAEKS

SEQ ID NO: 8 - protein, 807 aa, Canis lupus
MKRIRSIYIFCSIAISVQGWASKLPEERELTTNCSNMSLRKIPADLTPTTTTLDLSYNLLSQLQSSDFRSVSKLKVLILCHNRIQELNIKIFEFNRELRYLDLSYNRLKIVTWYSLAGLRHLDLSFNDFDTVPICEETGNMSHLEILGLSGAKIQKSNFQKIAHLHLKTVFLGLRSLSHYEEGSLPILNTTKLHIVLPMNTNFWVLLHDGIKTSKILEMTNIDGKSQFASYGTQKNLTLENSKTSILLLNKVDLLWDDLLLIFQFVWHTSVECFQIQHLTFGGKVYLDHYSFDYSNTVMRTIKLEHVQFRIFYIPQERVYLLFTKMDIENLTISDAQMPYMLFPIYPTRFQYLNFANNILTDDLFKQPIQLPHLKTLILKGNKLETFSLVSFFANNTSLKHLDLSQNLLQHENGENCFWPETLITMNLSSNKFADSVFRCLPRNIQILDLNNNKIQTVPKDIIHLKSLQELNLAFNFLTDLPGCSHFRKLSILNIEMNLILSPSLDFFQSCQEVKILNAGRNPFRCTCELRDFIQLEEYSEGMMIGWLDSYICEYPLTLKGTLLKDVHLPELSCNTTLLIVTIVVIMLVLGMAVAFCCFYFDLPWYLRMLGQWTLQRIRKTTQEQLKRNVQFHVFIAYSEHDSTWVKHELIPNLEKKEKLICLHEGNFDPGKSIIENIMNCIEKSYKSIFVLSPNFVQSEWCHYELYFAHHSLFHENSDYIIFILLEPIPLYCIPTKYPKLKALMEKKAYLEWPKDRRICGLFWANLRAAINANLLETREMYELQTFVGLNEESQGSAISLIRTDCL

SEQ ID NO: 9 - protein, 1121 aa, Canis lupus
MRDTQREAGTQAEGEAGSLQGARCGTRSQDPGITPWAEGRRSTPEPPRSPQPLCSALFNQTSVFHGGRRLKMVFPMWTLKRQFFILLNIILISKLLGARWFPKTLPCDVSLDAPKAHVIVDCTDKHLTEIPGGIPSNATNLTLTINHIPGISPASFHQLDYLVEIDFRCNCIPVRLGPKDHLCTRRPQIKPRSFSSLTYLKSLYLDGNQLLEIPEGLPPSLELLSLEANSIFSIMKNNLTELTNIERLYLGQNCYFRNPCNVSFFIEKDAFLSLKNLKLLSLKDNNITYVPTTLPSTLTELYLYNNAIAKIQEDDFNNLNQLRILDLSGNCPRCYNVPFPCTPCENNSPLQIHESAFDALTELQVLRLHSNSLQRVPQRWFKNIKKLKELDLSQNFLAKEIGDAKFLYLLHDLVQLDLSFNYELQVYRAALNLSDAFSSLKNLKVLRIKGYVFKELSSHHLSPLQSLTNLEVLDLGTNFIKIADLSIFEQFKTLKVIDLSMNKISPSGDSGEVGFCSSTRTSVEGHAPQVLETLHYFRYDEYARSCRFKNKETPSFLPFNKDCYMYGQTLDLSRNNIFFIKSSDFQHLSFLKCLNLSGNTIGQTLNGSEFQPLVELKYLDFSNNRLDLLYSTAFEELRKLEVLDISSNSHYFQSEGITHMLNFTKNLKVLKKLMMNNNDIATSTSRTMESESLKILEFRGNHLDVLWRDGDNRYLKFFKNLLNLEELDISENSLSFLPSGVFDGMPPNLKTLSLVKNGLKSFHWERLQYLKNLETLDLSYNELKIVPERLYNCSRSLKKLILKYNQIRQLTKHFLQDAFQLRYLDLSSNKIQIIQKTSFPENVLNNLEMLLLHHNRFLCTCDAVWFVWWVNHTEVTIPYLATDVTCVGPGAHKGQSVVSLDLYTCELDLTNLVLFSFSLSLALFLMVITTANHLYFWDVWYSYHYCKAKIKGYRRLKSLDSCYDAFVVYDTKDPAVTEWVLDELVAKLEDPREKHFNLCLEERDWLPGQPVLENLSQSIQLSKKTVFVMTNKYAKTENFKIAFYLSHQRLMDEKVDVIILIFLEKPLQKSKFLQLRKRLCKSSVLEWPRNPQAHPYFWQCLKNALATDNHVTYSQVFKETV

SEQ ID NO: 10 - protein, 1032 aa, Canis lupus
MGPCRGALHPLSLLVQAAALALALAQGTLPAFLPCELQPHGLVNCNWLFLKSVPRFSAAAPRGNVTSLSLYSNRIHHLHDYDFVHFVHLRRLNLKWNCPPASLSPMHFPCHMTIEPNTFLAVPTLEDLNLSYNSITTVPALPSSLVSLSLSRTNILVLDPATLAGLYALRFLFLDGNCYYKNPCQQALQVAPGALLGLGNLTHLSLKYNNLTVVPRGLPPSLEYLLLSYNHIITLAPEDLANLTALRVLDVGGNCRRCDHARNPCRECPKGFPQLHPNTFGHLSHLEGLVLRDSSLYSLDPRWFHGLGNLMVLDLSENFLYDCITKTKAFYGLARLRRLNLSFNYHKKVSFAHLHLASSFGSLLSLQELDIHGIFFRSLSKTTLQSLAHLPMLQRLHLQLNFISQAQLSIFGAFPGLRYVDLSDNRISGAAEPAAATGEVEADCGERVWPQSRDLALGPLGTPGSEAFMPSCRTLNFTLDLSRNNLVTVQPEMFVRLARLQCLGLSHNSISQAVNGSQFVPLSNLRVLDLSHNKLDLYHGRSFTELPRLEALDLSYNSQPFSMRGVGHNLSFVAQLPALRYLSLAHNGIHSRVSQQLRSASLRALDFSGNTLSQMWAEGDLYLRFFQGLRSLVQLDLSQNRLHTLLPRNLDNLPKSLRLLRLRDNYLAFFNWSSLALLPKLEALDLAGNQLKALSNGSLPNGTQLQRLDLSGNSIGFVVPSFFALAVRLRELNLSANALKTVEPSWFGSLAGALKVLDVTANPLHCACGATFVDFLLEVQAAVPGLPSRVKCGSPGQLQGRSIFAQDLRLCLDEALSWVCFSLSLLAVALSLAVPMLHQLCGWDLWYCFHLCLAWLPRRGRRRGVDALAYDAFVVFDKAQSSVADWVYNELRVQLEERRGRRALRLCLEERDWVPGKTLFENLWASVYSSRKTLFVLARTDRVSGLLRASFLLAQQRLLEDRKDVVVLVILCPDAHRSRYVRLRQRLCRQSVLLWPHQPSGQRSFWAQLGTALTRDNRHFYNQNFCRGPTTA

SEQ ID NO: 11 - DNA, 15 bp, Canis lupus
ctccttcgagaaagc

SEQ ID NO: 12 - DNA, 18 bp, Canis lupus
ttatcgtgaagatcaagc

SEQ ID NO: 13 - DNA, 18 bp, Canis lupus
catcatctgggtaacgaa

SEQ ID NO: 14 - DNA, 20 bp, Canis lupus
catttgaaattgagaaatac

SEQ ID NO: 15 - DNA, 19 bp, Canis lupus
acaccaagtcgttagtaat

SEQ ID NO: 16 - DNA, 14 bp, Canis lupus
cccgccgtgagcag

SEQ ID NO: 17 - DNA, 16 bp, Canis lupus
tctccaagagcttctg

SEQ ID NO: 18 - DNA, 18 bp, Canis lupus
caaaactgcaggtgctgg

SEQ ID NO: 19 - DNA, 14 bp, Canis lupus
cccatgcgggaaat

SEQ ID NO: 20 - DNA, 20 bp, Canis lupus
aagttattccgagtaagatt

SEQ ID NO: 21 - DNA, 15 bp, Canis lupus
ttccccgaagccctg

SEQ ID NO: 22 - DNA, 17 bp, Canis lupus
tgtttgccgtcactcaa

SEQ ID NO: 23 - DNA, 14 bp, Canis lupus
tccgggccgtcacc

SEQ ID NO: 24 - DNA, 13 bp, Canis lupus
aggcgcaacaaag

SEQ ID NO: 25 - DNA, 15 bp, Canis lupus
tccttccgggagctg

SEQ ID NO: 26 - DNA, 14 bp, Canis lupus
ctcaaccggatccc

SEQ ID NO: 27 - DNA, 13 bp, Canis lupus
tcgcacggcttcg

SEQ ID NO: 28 - DNA, 14 bp, Canis lupus
agcccccggaacac

SEQ ID NO: 29 - DNA, 14 bp, Canis lupus
tcaggtcgaggccc

SEQ ID NO: 30 - DNA, 13 bp, Canis lupus
cgcagcgcacgtc

SEQ ID NO: 31 - DNA, 15 bp, Canis lupus
ctcgtggccgccaag

SEQ ID NO: 32 - DNA, 14 bp, Canis lupus
ccaggtgggacgcg

SEQ ID NO: 33 - DNA, 15 bp, Canis lupus
cctggacggcgccct

SEQ ID NO: 34 - DNA, 14 bp, Canis lupus
cagcgccggtactt

SEQ ID NO: 35 - DNA, 12 bp, Canis lupus
cgcgcgcccctc

SEQ ID NO: 36 - DNA, 18 bp, Canis lupus
ttcttttgcataataacc

SEQ ID NO: 37 - DNA, 15 bp, Canis lupus
agggagcctgcttct

SEQ ID NO: 38 - DNA, 14 bp, Canis lupus
tgcatcggaaatcg

SEQ ID NO: 39 - DNA, 18 bp, Canis lupus
cttgagaagccccttcag

SEQ ID NO: 40 - DNA, 14 bp, Canis lupus
ccctccggaagatt

SEQ ID NO: 41 - DNA, 18 bp, Canis lupus
ttgtccacttcgtccacc

SEQ ID NO: 42 - DNA, 15 bp, Canis lupus
cgctcagcaagacca

SEQ ID NO: 43 - DNA, 16 bp, Canis lupus
cccagtgggcccagag

SEQ ID NO: 44 - DNA, 15 bp, Canis lupus
acggctaggcaggcc

SEQ ID NO: 45 - DNA, 12 bp, Canis lupus
cccgccgccgcc

SEQ ID NO: 46 - DNA, 16 bp, Canis lupus
ccacagccatccccag

SEQ ID NO: 47 - DNA, 16 bp, Canis lupus
ccaggaggaacagcgt

SEQ ID NO: 48 - protein, 10 aa, Canis lupus
TINAFLKEQL

SEQ ID NO: 49 - protein, 130 aa, Canis lupus
  VARIANT (7)..(7): Xaa is an amino acid selected from Ile and Phe
  VARIANT (129)..(129): Xaa is an amino acid selected from Pro and Leu
KVKVLDXHDNRIRSIPKPIMKLEDLQELNVASNSLAHFPDCGTFNRLSVLIIDSNSISNPSADFLQSCHNIRSMSAGNNPFQCTCELREFVQSLGQVASKVVEGWPDSYKCDSPENYKGTLLKDFHVSXL

SEQ ID NO: 50 - protein, 10 aa, Canis lupus
QNNIIWATKK

SEQ ID NO: 51 - protein, 10 aa, Canis lupus
GVTLFLLAVL

SEQ ID NO: 52 - DNA, 14 bp, Canis lupus
cccagcttgcttgc

SEQ ID NO: 53 - DNA, 25 bp, Artificial sequence
  Artificially synthesized forward primer
agttggtttggcatacaagcataga

SEQ ID NO: 54 - DNA, 25 bp, Artificial sequence
  Artificially synthesized reverse primer
tctgaagtcagggtaaccttgtagt

SEQ ID NO: 55 - DNA, 24 bp, Artificial sequence
  Artificially synthesized forward primer
gacacttcactgaaggccttatct

SEQ ID NO: 56 - DNA, 37 bp, Artificial sequence
  Artificially synthesized reverse primer
agattttatagatataactttgtggcaaattgaatgc

SEQ ID NO: 57 - DNA, 30 bp, Artificial sequence
  Artificially synthesized forward primer
ccaccttacaaattatgagaatcagcagaa

SEQ ID NO: 58 - DNA, 23 bp, Artificial sequence
  Artificially synthesized reverse primer
agcctgtgaaaggaatccagttg

SEQ ID NO: 59 - DNA, 18 bp, Artificial sequence
  Artificially synthesized forward primer
gccgtgtgctgtgtcttg

SEQ ID NO: 60 - DNA, 18 bp, Artificial sequence
  Artificially synthesized reverse primer
acagcccgtggaaatggt

SEQ ID NO: 61 - DNA, 24 bp, Artificial sequence
  Artificially synthesized forward primer
aagttcttcacgcctcagtacatt

SEQ ID NO: 62 - DNA, 27 bp, Artificial sequence
  Artificially synthesized reverse primer
attaggaagtcagctccaattggaaaa

SEQ ID NO: 63 - DNA, 22 bp, Artificial sequence
  Artificially synthesized forward primer
cagccattgcttctccaacttc

SEQ ID NO: 64 - DNA, 24 bp, Artificial sequence
  Artificially synthesized reverse primer
cttgggcaatgtgtaaaagctcat

SEQ ID NO: 65 - DNA, 25 bp, Artificial sequence
  Artificially synthesized forward primer
agatggcaacggttggaaatagtta

SEQ ID NO: 66 - DNA, 25 bp, Artificial sequence
  Artificially synthesized reverse primer
ggaagtgagaacaaactccttgaga

SEQ ID NO: 67 - DNA, 25 bp, Artificial sequence
  Artificially synthesized forward primer
gaacaaggacaacagcattttccaa

SEQ ID NO: 68 - DNA, 23 bp, Artificial sequence
  Artificially synthesized reverse primer
tctgatgttcacagtcacaagca

SEQ ID NO: 69 - DNA, 24 bp, Artificial sequence
  Artificially synthesized forward primer
cacgttctccctcttcatcttctc

SEQ ID NO: 70 - DNA, 17 bp, Artificial sequence
  Artificially synthesized reverse primer
caaaggccccggagctt

SEQ ID NO: 71 - DNA, 19 bp, Artificial sequence
  Artificially synthesized forward primer
gtatcctccgcctgccatt

SEQ ID NO: 72 - DNA, 24 bp, Artificial sequence
  Artificially synthesized reverse primer
ttaccttgaggagagtctgtttgc

SEQ ID NO: 73 - DNA, 18 bp, Artificial sequence
  Artificially synthesized forward primer
ctccgcctgccattttcc

SEQ ID NO: 74 - DNA, 25 bp, Artificial sequence
  Artificially synthesized reverse primer
ggtccttgagaaacacttaccttga

SEQ ID NO: 75 - DNA, 22 bp, Artificial sequence
  Artificially synthesized forward primer
ctcctgctgagcttcaactaca

SEQ ID NO: 76 - DNA, 15 bp, Artificial sequence
  Artificially synthesized reverse primer
gcagccgctccagga

SEQ ID NO: 77 - DNA, 25 bp, Artificial sequence
  Artificially synthesized forward primer
cgtgttgacagacggttatttcaga

SEQ ID NO: 78 - DNA, 23 bp, Artificial sequence
  Artificially synthesized reverse primer
ctcgaggctcccaatctgatttt

SEQ ID NO: 79 - DNA, 18 bp, Artificial sequence
  Artificially synthesized forward primer
gggagcctcgagcttcac

SEQ ID NO: 80 - DNA, 16 bp, Artificial sequence
  Artificially synthesized reverse primer
acacgcagccgggatc

SEQ ID NO: 81 - DNA, 18 bp, Artificial sequence
  Artificially synthesized forward primer
gctgggttccctgaggtc

SEQ ID NO: 82 - DNA, 16 bp, Artificial sequence
  Artificially synthesized reverse primer
agggagagcgccttgc

SEQ ID NO: 83 - DNA, 17 bp, Artificial sequence
  Artificially synthesized forward primer
tgctgcggctggatctg

SEQ ID NO: 84 - DNA, 18 bp, Artificial sequence
  Artificially synthesized reverse primer
gggcgttcagggagaaga

SEQ ID NO: 85 - DNA, 14 bp, Artificial sequence
  Artificially synthesized forward primer
gggcggtgctggga

SEQ ID NO: 86 - DNA, 18 bp, Artificial sequence
  Artificially synthesized reverse primer
tgtggttcaggtgcagca

SEQ ID NO: 87 - DNA, 16 bp, Artificial sequence
  Artificially synthesized forward primer
gacctcacggcgctga

SEQ ID NO: 88 - DNA, 17 bp, Artificial sequence
  Artificially synthesized reverse primer
gcgtgctcagcctgttg

SEQ ID NO: 89 - DNA, 18 bp, Artificial sequence
  Artificially synthesized forward primer
gtggctcaaccggaccaa

SEQ ID NO: 90 - DNA, 19 bp, Artificial sequence
  Artificially synthesized reverse primer
agccctccatggagacaga

SEQ ID NO: 91 - DNA, 21 bp, Artificial sequence
  Artificially synthesized forward primer
cctcttcatcttctccaccgt

SEQ ID NO: 92 - DNA, 23 bp, Artificial sequence
  Artificially synthesized reverse primer
gccttgtaacagaggaagcaaag

SEQ ID NO: 93 - DNA, 16 bp, Artificial sequence
  Artificially synthesized forward primer
tgcctggaggccttcg

SEQ ID NO: 94 - DNA, 18 bp, Artificial sequence
  Artificially synthesized reverse primer
cacgaccaccaggacgag

SEQ ID NO: 95 - DNA, 16 bp, Artificial sequence
  Artificially synthesized forward primer
tgcctggaggccttcg

SEQ ID NO: 96 - DNA, 17 bp, Artificial sequence
  Artificially synthesized reverse primer
cccacgaccaccaggac

SEQ ID NO: 97 - DNA, 15 bp, Artificial sequence
  Artificially synthesized forward primer
aggcacccggccatc

SEQ ID NO: 98 - DNA, 18 bp, Artificial sequence
  Artificially synthesized reverse primer
ccacgtcctgcagatcct

SEQ ID NO: 99 - DNA, 18 bp, Artificial sequence
  Artificially synthesized forward primer
acgctctcccgacacatc

SEQ ID NO: 100 - DNA, 14 bp, Artificial sequence
  Artificially synthesized reverse primer
cgcagcgggatgcc

SEQ ID NO: 101 - DNA, 26 bp, Artificial sequence
  Artificially synthesized forward primer
gttttcagatgtttacctcccaaggt

SEQ ID NO: 102 - DNA, 26 bp, Artificial sequence
  Artificially synthesized reverse primer
agcttcatgattggtttaggaatgct

SEQ ID NO: 103 - DNA, 22 bp, Artificial sequence
  Artificially synthesized forward primer
atctgctaccaattgctcattt

SEQ ID NO: 104 - DNA, 33 bp, Artificial sequence
  Artificially synthesized reverse primer
agtgtttttgtatctagaatttgaagacttcct

SEQ ID NO: 105 - DNA, 15 bp, Artificial sequence
  Artificially synthesized forward primer
tcccgcgcagacgtg

SEQ ID NO: 106 - DNA, 19 bp, Artificial sequence
  Artificially synthesized reverse primer
agccctccatggagacaga

SEQ ID NO: 107 - DNA, 19 bp, Artificial sequence
  Artificially synthesized forward primer
agagagaggcagggacaca

SEQ ID NO: 108 - DNA, 19 bp, Artificial sequence
  Artificially synthesized reverse primer
ggatcgagtcccacatcgg

SEQ ID NO: 109 - DNA, 24 bp, Artificial sequence
  Artificially synthesized forward primer
ccagctggactatctggtagagat

SEQ ID NO: 110 - DNA, 22 bp, Artificial sequence
  Artificially synthesized reverse primer
ccccagtcgaacaggtatacag

SEQ ID NO: 111 - DNA, 25 bp, Artificial sequence
  Artificially synthesized forward primer
tcatggatgaaaaagtggacgtcat

SEQ ID NO: 112 - DNA, 22 bp, Artificial sequence
  Artificially synthesized reverse primer
ggagctggaggaacttggattt

SEQ ID NO: 113 - DNA, 25 bp, Artificial sequence
  Artificially synthesized forward primer
ggtctttgaaggaacttagcctgat

SEQ ID NO: 114 - DNA, 22 bp, Artificial sequence
  Artificially synthesized reverse primer
cgtgagcccagacgtattcttt

SEQ ID NO: 115 - DNA, 24 bp, Artificial sequence
  Artificially synthesized forward primer
gcttcgctacctaaacctctctag

SEQ ID NO: 116 - DNA, 22 bp, Artificial sequence
  Artificially synthesized reverse primer
catgttgtcaaaccacgttgca

SEQ ID NO: 117 - DNA, 23 bp, Artificial sequence
  Artificially synthesized forward primer
ccaccacctccatgactatgact

SEQ ID NO: 118 - DNA, 23 bp, Artificial sequence
  Artificially synthesized reverse primer
ggcagttccacttgagattgaga

SEQ ID NO: 119 - DNA, 24 bp, Artificial sequence
  Artificially synthesized forward primer
gctggacatacatggcatcttctt

SEQ ID NO: 120 - DNA, 15 bp, Artificial sequence
  Artificially synthesized reverse primer
ggtgggccagcgact

SEQ ID NO: 121 - DNA, 20 bp, Artificial sequence
  Artificially synthesized forward primer
ggagagagtctggccacagt

SEQ ID NO: 122 - DNA, 18 bp, Artificial sequence
  Artificially synthesized reverse primer
cggcatgaaggcctctga

SEQ ID NO: 123 - DNA, 16 bp, Artificial sequence
  Artificially synthesized forward primer
gctggaggtgcaggct

SEQ ID NO: 124 - DNA, 16 bp, Artificial sequence
  Artificially synthesized reverse primer
ggctgccgcacttgac

SEQ ID NO: 125 - DNA, 15 bp, Artificial sequence
  Artificially synthesized forward primer
gtgcctggcctggct

SEQ ID NO: 126 - DNA, 20 bp, Artificial sequence
  Artificially synthesized reverse primer
gccttgtcgaagaccacgaa

SEQ ID NO: 127 - DNA, 25 bp, Artificial sequence
  Artificially synthesized forward primer
tctgttgattgtcaccattgtggtt

SEQ ID NO: 128 - DNA, 25 bp, Artificial sequence
  Artificially synthesized reverse primer
cagatcaaagtagaagcagcagaag

(19) United States
(12) Patent Application Publication
(10) Pub. No.: US 2017/0233811 A1
(43) Pub. Date: Aug. 17, 2017

(54) DNA ARRAY FOR DETECTING CANINE TOLL-LIKE RECEPTOR GENE MUTATIONS

(71) Applicants: Universitat Autonoma de Barcelona, Bellaterra Cerdanyola del Valles (ES); Crag-Centre de Recerca en Agrigenòmica, Bellaterra Cerdanyola (ES)

(72) Inventors: Armand Sanchez Bonastre, Bellaterra Cerdanyola del Valles (ES); Olga Francino Marti, Bellaterra Cerdanyola del Valles (ES); Anna Maria Cusco Marti, Bellaterra Cerdanyola (ES)

(21) Appl. No.: 15/315,016

(22) PCT Filed: May 29, 2015
(86) PCT No.: PCT/EP2015/061985

§ 371 Date: Nov. 30, 2016

(30) Foreign Application Priority Data
May 30, 2014 (EP) 14170516.0

(51) Int. Cl. C12Q 1/68

(52) U.S. Cl. CPC C12Q 1/6883; C12Q 2600/16; C12Q 2600/118; C12Q 2600/156

(57) ABSTRACT

DNA array for detecting canine toll-like receptor gene mutations New mutations in canine toll-like receptor genes are provided. The invention relates further to DNA arrays comprising oligonucleotides capable of hybridizing to at least a TLR gene fragment codifying for at least one of the new mutations leading to non-functional proteins. The invention also includes methods for the analysis of mutations and for the genetic predisposition of an individual of Canis genera to suffer from any disease related with dysfunctions of the innate immunity system. The invention also provides a method for the individual identification of canine subjects.

Present invention relates to meaningful mutations identified in some critical genes involved in the innate immunity in dogs (canine genus), as well as to canine disease diagnosis, prediction of canine disease susceptibility, and to the individual identification of canine subjects.

### BACKGROUND ART

Increasing number of domestic animals, in particular dogs and cats, has promoted the study, particular diagnostic, and particular treatments of these animals. For the study of the diseases is interesting to know how the immune system functions in these animals, particularly when infectious or autoimmune disease are to be faced. Besides, other systems and metabolic pathways are studied in order to determine if they are specific of species or not. Additionally, and considering that dogs include the greatest number of breeds known in the animal kingdom, there can be breed specific diseases or susceptibilities which may influence the final diagnostic and treatment applied to specific breeds.

Despite it is known that some dog breeds have special susceptibilities to diseases and to particular treatments, little is known about the causes of these facts and, although it is widely accepted that genomic profiles are probably the greatest cause, poor meaningful studies have been performed. In part, this lack of knowledge is due to the lack of appropriate tools for the study of the dog genome or Canis lupus familiaris genome.

In the specific case of susceptibilities to some diseases, dog immune response has to be one of the objects for next years.

Besides, appropriate unequivocal identification of individuals or appropriate classification of any individual to a particular group would be also an aim.

Toll-like receptors (TLRs) are the most widely studied pattern recognition receptors (PRRs) and are considered to be the primary sensors of pathogens in innate immunity. These molecules are constituted by leucine-rich repeat (LRR) domains, a unique intramembrane domain and a Toll/Interleukin-1 receptor (TIR) domain. Pathogen-associated molecular Patterns (PAMPs) are sensed through LRR domain, and signals are transduced through TIR domain, which is always located in the cytoplasm, in order to activate innate immunity response.

10 TLRs have been identified in dogs. They are classified in two groups, depending on their location and the PAMPs detected. TLR which target nucleic acids (TLR3, 7, 8 and 9) are located in endosomal membranes and are also known as viral TLRs. On the other hand, there are membrane TLRs (TLR1, 2, 4, 5 and 6) which detect components of extracellular pathogens (bacteria, fungi, etc). TLR10 is also a membrane TLR, however its ligand is not yet known.

Still today little is known about canine TLR and its role or implication in diseases, basically because, as above stated, there are no tools to test for relevant mutations. Moreover, differences among breeds have been explored poorly. Kathrani et al., in “Polymorphisms in the Tlr4 and Tlr5 Gene Are Significantly Associated with Inflammatory Bowel Disease in German Shepherd Dogs”, PLoS ONE—2010, Vol. No. 5 (12), pp.: 1-10, associated polymorphisms in TLR4 and TLR5 with Inflammatory Bowel disease (IBD) in German shepherd dogs (GSD). They concluded that further studies were required to confirm the functional importance of the mutations. The same Kathrani et al. showed that only some protective SNPs from TLR5 were associated with IBD in other 38 dog breeds (Kathrani et al., “Breed-independent toll-like receptor 5 polymorphisms show association with canine inflammatory bowel disease”, Tissue Antigens—2011, Vol. No. 78, pp.: 94-101). All the mutations mentioned by Kathrani have been identified by sequencing TLR genes of dogs with IBD and then comparing sequence data to the canine genome.

Although no other polymorphisms have been associated to illness in dogs until date, some studies have reported differential expression of some TLRs related to inflammatory or infectious diseases, such as TLR2 in IBD; TLRs 2, 4, 5 and 9 in chronic enteropathies in German Shepherd; TLR4 in osteoarthritis and in infected canine endometrium; TLRs 1-4, 6-10 in sino-nasal aspergillosis and idiopathic lymphoplasmacytic rhinitis; and TLR2 and TLR9 in Leishmania infected dogs.

Therefore, there is still a need of methods and tools to better characterize physiology and immunity response profiles in domestic animals, in particular in dogs.

In other animals of economic interest, such as cattle, much more information of TLR polymorphisms has been significantly associated. For example, predisposition to mastitis or to natural bacterial infections (Kannaki et al., “Toll-like receptors and their role in animal reproduction”, Animal Reproducing Science-2011, Vol. No. 125 (1-4), pp.:1-12).

On the other hand, nowadays identification of dogs (or other animals) is a complex task, finally carried out with DNA technologies as in humans. Nonetheless, analysis of all the genome is always tedious and expensive. Therefore, there is also a need of new tools for unequivocally identifying individuals, as well as for the traceability of their waste material, or other samples from them.

There are publications disclosing DNA arrays for canine genome analysis. An example of these is the document of Ke et al., “Assessment of the functionality of genome-wide canine SNP arrays and implications for canine disease association studies”, Animal Genetics—2010, Vol. No. 42(2), pp.:181-190. In this document the Illumina 22K canine SNP array and the Affymetrix 50K canine SNP array are used for assessing in different breeds SNP and the linkage disequilibriums (LD) associated thereto. At this respect the document concludes that the study designs should be carefully assessed for individual breeds before genome-wide association studies are performed. Both arrays are complex and expensive for the purpose of individual identification and a reliable distinction among two or more animals.

### SUMMARY OF THE INVENTION

The inventors determined new mutations, some of them being single nucleotide polymorphisms (SNP) in canine TLR genes. In addition they have developed a method and an array for determining the innate immunity profile of individuals of the Canis genera based on these new mutations, which are mainly causing protein disruption or loss of function. These mutations, mainly non-synonymous mutations and frameshift mutations may impair, alter or affect the innate immunity response in individuals of the Canis genera, thus including among other dogs and wolves.

Noteworthy, most of individuals (i.e. dogs) with innate immune system defects have intact adaptive immune systems with normal immunoglobulins, antibodies and T-cells. A type of white blood cell called an eosinophil may be increased in the blood; elevated IgE immunoglobulin levels may also be present. The diagnosis of defects or dysfunctions in innate immunity system (response) is usually made by measuring cytokine production by white blood cells, activated by microbial products that stimulate the cells. Testing of TLR function is becoming available through commercial reference laboratories, such as the one followed by ARUP LABORATORIES® with TLR ligands. Abnormal tests need to be confirmed by repeat testing. Persistently abnormal tests may be followed up by specific genetic tests.

Using informatic tools for predicting the effect of the new mutations in the proteins (gene products) codified by the canine tlr genes, inventors identified those changes leading to a loss of protein function, damaging amino acid changes, or deleterious mutations that are involved in the loss of innate immunity response in individuals of the Canis genera. These mutations are also responsible of certain susceptibilities to diseases or of treatment resistances associated with an impaired innate immunity.

New mutations correspond mainly to the following amino acid changes in the protein sequences of canine toll-like receptors:

a change of a serine (S) by a leucine (L) at position 516 of canine TLR2 polypeptide of SEQ ID NO: 1; a change of a phenylalanine (F) by a cysteine (C) at position 888 of canine TLR5 polypeptide of SEQ ID NO: 2; a change of a leucine (L) by a phenylalanine (F) at position 457 of canine TLR6 polypeptide of SEQ ID NO: 3; and a change of a valine (V) by an alanine (A) at position 157 of canine TLR8 polypeptide of SEQ ID NO: 4.

The invention relates to a DNA array comprising canine toll-like receptor (TLR) gene fragments.

Thus, in a first aspect the invention relates to DNA arrays comprising:

(i) a canine toll-like receptor 2 (tlr 2) gene fragment that in the entire nucleotide sequence of the gene codifies for a change of a serine (S) by a leucine (L) at position 516 of canine TLR2 polypeptide of SEQ ID NO: 1;

(ii) a canine toll-like receptor 5 (tlr 5) gene fragment that in the entire nucleotide sequence of the gene codifies for a change of a phenylalanine (F) by a cysteine (C) at position 888 of canine TLR5 polypeptide of SEQ ID NO: 2;

(iii) a canine toll-like receptor (tlr 6) gene fragment that in the entire nucleotide sequence of the gene codifies for a change of a leucine (L) by a phenylalanine (F) at position 457 of canine TLR6 polypeptide of SEQ ID NO: 3; and

(iv) a canine toll-like receptor 8 (tlr 8) gene fragment that in the entire nucleotide sequence of the gene codifies for a change of a valine (V) by an alanine (A) at position 157 of canine TLR8 polypeptide of SEQ ID NO: 4.

Therefore, the DNA arrays comprise canine toll-like receptor tlr gene fragments codifying for peptides, said peptides in the entire corresponding TLR amino acid sequences having any of the following amino acid changes:

(i) A change of a serine (S) by a leucine (L) at position 516 of canine TLR2 polypeptide of SEQ ID NO: 1;

(ii) A change of a phenylalanine (F) by a cysteine (C) at position 888 of canine TLR5 polypeptide of SEQ ID NO: 2;

(iii) A change of a leucine (L) by a phenylalanine (F) at position 457 of canine TLR6 polypeptide of SEQ ID NO: 3; and

(iv) A change of a valine (V) by an alanine (A) at position 157 of canine TLR8 polypeptide of SEQ ID NO: 4.

These DNA arrays comprise oligonucleotide sequences that are fragments of canine tlr genes, which fragments in the entire gene will give raise to mutated proteins. These oligonucleotides of the array are indeed complementary to canine toll-like receptor gene fragments (being the codifying or complementary strain of the gene) present in an isolated sample from a subject of the Canis genera. The oligonucleotides are capable of detecting the above-identified mutations by hybridizing to these gene fragments codifying for the mutations, and thus making the mutations detectable. Hybridization will take place under stringent conditions, preferably at 60° C., and in any case in a range from 50° C. to 70° C. Particular TaqMan® probe hybridization conditions can be seen in http://premierbiosoft.com/tech_notes/TaqMan.html (retrieved in Apr. 23, 2014)

Thus, this first aspect could also be formulated as DNA arrays comprising oligonucleotides which are complementary and capable of hybridizing at a temperature of 60° C. (50-70° C.) to canine toll-like receptor gene fragments from a sample, the tlr gene fragments of the sample codifying, when forming part of the entire corresponding gene, for at least one of the mutations indicated above from (i) to (iv).

By means of these arrays, at least five critical mutations are detected in a simple and fast way, and allow obtaining a genomic profile with important information regarding innate immunity due to the fact that the identified mutations lead to a loss of protein function and/or to an altered protein structure. Indeed, the arrays allow compiling important information of several proteins/genes which, if mutated, will in addition probably lose its function and/or structure.

Leading then with the advantages of the arrays, a second aspect of the invention is a method for the identification of mutations in canine toll-like receptor genes, the method comprising:

adding an isolated animal sample comprising DNA to a DNA array as defined above; and

determining if the canine toll-like receptor gene fragments hybridize with sample gene fragments.

This method represents a simple way to detect if an isolated sample comprising genetic information (i.e polynucleotide data, namely DNA data) includes tlr gene fragments carrying any of the newly identified critical mutations. Genetic information may be retrieved from the DNA of the sample as well as from cDNA in case the starting material is RNA, which is firstly converted to cDNA by any routinely methods such as the Reverse Transcription Polymerase Chain Reaction (RT-PCR).

Detection of these particular non-synonymous mutations in a TLR proteins (namely in canine TLR2, TLR5, TLR6 and TLR8) have impact on protein function and/or structure and alters or modifies the innate immunity system (response) of the animal. This modification of the final innate immunity response will be informing about the predisposition of suffering from particular diseases. On the other hand, detection of mutated TLR with impact on the protein function serves as basis for determining the cause of a particular disease yet manifested in a canine individual. The combined detection of these five mutations represents meaningful and precise information. It is widely accepted that the final result of a response (i.e. innate immunity response) derives from all the proteins and compounds intervening in it. Therefore, with the detection or determination of many of the critical mutations a more reliable data of this final result can be obtained.

On the other hand, the detection of a particular frameshift mutation in a tlr gene will also have impact on protein function and on the innate immunity system (response) of the animal.

The critical mutations in each of canine TLR2, TLR5, TLR6 and TLR8, were detected in animals (dogs and wolves) some of them suffering from leishmaniosis, and demodicosis. In particular, mutation S516L in SEQ ID NO: 1 (canine TLR2) has been detected in German Shepherd Dog and in French Bulldog in cohorts with leishmaniosis, and/or demodicosis. The mutation V157A in SEQ ID NO: 4 (TLR8) is present in Yorkshire and French Bulldog in a cohort with demodicosi. Therefore, an application of the array of the invention derives from allowing determination of some critical mutations that may be shared in a particular animal cohort. This knowledge helps in the development of specific therapies or assays in homogenized animal cohorts. Besides, the array is also useful in the detection of particular susceptibilities to diseases.

With regard to critical mutations in TLR2 of SEQ ID NO: 1, an aspect of the invention is a peptide sequence comprising SEQ ID NO: 48 (TINAFLKEQL), as well as an oligonucleotide codifying for said SEQ ID NO: 48. This peptide sequence is a fragment of the TLR2 polypeptide of SEQ ID NO: 1, having a change of a serine (S) by a leucine (L) at position 516 of this SEQ ID NO: 1. This is a deleterious mutation (according to Provean informatics tool), and affects the function of the protein according to SIFT bioinformatics tool, and still is a probably damaging mutation according to the bioinformatic predicting tool Polyphen-2.

With regard to critical mutations in TLR6 of SEQ ID NO: 3, an aspect of the invention is a peptide sequence comprising SEQ ID NO: 49, (KVKVLDX¹HDN RIRSIPKPIM KLEDLQELNV ASNSLAHFPD CGTFNRLSVL IIDSNSISNP SADFLQSCHN IRSMSAGNNP FQCTCELREF VQSLGQVASK VVEGWPDSYK CDSPENYKGT LLKDFHVSX²L), wherein X¹ is selected from I and F, and X² is selected from P and L, with the proviso that at least one of X¹ or X² is, respectively, F or L. Another aspect is further an oligonucleotide codifying for said SEQ ID NO: 49. This peptide sequence is a fragment of canine TLR6 polypeptide of SEQ ID NO: 3 having the change of a leucine (L) by a phenylalanine (F) at position 457 and a change of a proline (P) by a leucine (L) at position 579 of canine of this SEQ ID NO: 3. Both are deleterious mutations, according to Provean informatics tool, affect the function of the protein according to SIFT bioinformatics tool, and still are probably damaging mutations according to the bioinformatic predicting tool Polyphen-2.

With regard to critical mutations in TLR8 of SEQ ID NO: 4, an aspect of the invention is a peptide sequence comprising SEQ ID NO: 50 (QNNIIWATKK), as well as an oligonucleotide codifying for said SEQ ID NO: 50. This peptide sequence is a fragment of canine TLR8 polypeptide of SEQ ID NO: 4 having the change of a valine (V) by an alanine (A) at position 157 of SEQ ID NO: 4. It is a deleterious mutations, according to Provean informatics tool, it affects the function of the protein according to SIFT bioinformatics tool, and still is a probably damaging mutation according to the bioinformatic predicting tool Polyphen-2.

With regard to critical mutations in TLR5 of SEQ ID NO: 2, an aspect of the invention is a peptide sequence comprising SEQ ID NO: 51 (GVTLFLLAVL), as well as an oligonucleotide codifying for said SEQ ID NO: 51. This peptide sequence is a peptide fragment of canine TLR5 of SEQ ID NO: 2, that includes the change of a leucine (L) by a phenylalanine (F) at position 888 of of this SEQ ID NO: 2

Bioinformatic tools, Provean, SIFT and Polyphen-2 used in the present invention are referenced in the examples.

Therefore, another aspect of the invention is an in vitro method for the analysis of the genetic predisposition of a dog to suffer from any disease related with dysfunctions of the innate immunity system, the method comprising carrying out the method as defined above, including the detection of particular tlr gene mutations, and correlating any detected mutation with a disease.

Information from tlr gene mutations represents, indeed, an imprint or image of the innate immunity profile of an animal, in this case of a dog or a dog breed, generically in an individual of the Canis genera, including dogs (Canis lupus familiaris subspecies) and wolfs (Canis lupus species). Thus, another aspect of the invention is an in vitro method for the analysis of the canine innate immunity profile, the method comprising determining the presence or absence of canine toll-like receptor gene mutations by means of the DNA array as defined above.

This aspect can also be formulated as an in vitro method for the analysis of the canine innate immunity profile, the method comprising determining the presence or absence of canine toll-like receptor mutations selected from

(i) A change of a serine (S) by a leucine (L) at position 516 of canine TLR2 polypeptide of SEQ ID NO: 1;

(ii) A change of a phenylalanine (F) by a cysteine (C) at position 888 of canine TLR5 polypeptide of SEQ ID NO: 2;

(iii) A change of a leucine (L) by a phenylalanine (F) at position 457 of canine TLR6 polypeptide of SEQ ID NO: 3; and

(iv) A change of a valine (V) by an alanine (A) at position 157 of canine TLR8 polypeptide of SEQ ID NO: 4.

A global knowledge of the possible functionality status of innate immunity effectors (i.e., the proteins and routes involved in the response), allows predicting the most probable response type.

At the same time, knowing the innate immunity profile or, in other words, the genomic profile of this response by means of TLR sequences of an individual, permits classifying said individual to homogeneous groups with regard to these features (sequences of TLR). Homogeneous groups of dogs or wolves are important for the study of some metabolic routes or to perform clinical trials without the individual variability factor.

In the same way, knowing the TLR profile as such is useful not only to homogenize a test canine population for a particular assay, but also for performing prevalence studies of infectious diseases or microbiome studies, in which TLR may be involved.

Finally, the inventors have also proved that surprisingly TLR genes or the corresponding codified proteins may be used as a way to unequivocally identify Canis lupus genera individuals. Indeed, by determining some TLR mutations by means of the array as defined above, sufficient information is provided from an individual to distinct it from another one. This use of TLR genes opens the possibility of identifying waste material, or other samples from an individual and to correlate the sample with said individual. Thus, another aspect of the invention is an in vitro method for the individual identification of a Canis lupus genera animal, comprising determining the presence or absence of canine toll-like receptor mutations in an isolated sample by means of the DNA array as defined above; and establishing a combination of detected and non-detected mutations in said sample.

### DETAILED DESCRIPTION OF THE INVENTION

In the sense of the present invention, the term “canine” relates to the family of Canidae including domestic dogs, wolves, foxes, jackals, coyotes, and other dog-like mammals. This family includes the sub-family Caninae including in turn the genus Canis (including dogs, wolves, coyotes and jackals). Among the Canis species there is the Canis lupus (primarily represented by wolves) specie, with the sub-species Canis lupus familiaris (represented by domestic dogs). When canine is used in the present invention, it encompasses in particular the species and subspecies Canis lupus and Canis lupus familiaris. Thus, by “canine toll-like receptor (TLR) is to be understood the TLR of any of the individuals of the Canis genus, and in particular of the species Canis lupus (wolves) and Canis lupus familiaris (dogs).

Oligonucleotides are short (from 10 to 60 nucleotide monomers), single-stranded DNA or RNA molecules that have a wide range of applications in genetic testing, research, and forensics. Commonly made in the laboratory by solid-phase chemical synthesis, these small bits of nucleic acids can be manufactured with any user-specified sequence, and so are vital for artificial gene synthesis, polymerase chain reaction (PCR), DNA sequencing, library construction and as molecular probes. In nature, oligonucleotides are usually found as small RNA molecules that function in the regulation of gene expression (e.g. microRNA), or are degradation intermediates derived from the breakdown of larger nucleic acid molecules. Oligonucleotides are characterized by the sequence of nucleotide residues that make up the entire molecule. The length of the oligonucleotide is usually denoted by “-mer” (from Greek meros, “part”). For example, an oligonucleotide of six nucleotides (nt) is an hexamer, while one of 25 nt would usually be called a “25-mer”. Oligonucleotides readily bind, in a sequence-specific manner, to their respective complementary oligonucleotides, DNA, or RNA to form duplexes or, less often, hybrids of a higher order. This basic property serves as a foundation for the use of oligonucleotides as probes for detecting DNA or RNA. Examples of procedures that use oligonucleotides include DNA microarrays, Southern blots, ASO analysis, fluorescent in situ hybridization (FISH), and the synthesis of artificial genes. Oligonucleotides are also indispensable elements in antisense gene therapy.

In the sense of the present invention a “gene fragment that in the entire nucleotide sequence of the gene codifies for a change of amino acids”, for example, codifying for a change of a serine (S) by a leucine (L) at position 516 of canine TLR2 polypeptide of SEQ ID NO: 1, refers to a nucleic acid fragment, which when forming part of the complete tlr 2 gene, said fragment gives rise to a mutated protein in the indicated amino acid position after the transcription and translation of the entire gene. Usually, the gene fragments are oligonucleotides as above defined, thus from 10 to 30 nucleotides.

For “frameshift mutation” is to be understood as a genetic mutation caused by indels (insertions or deletions) of a number of nucleotides in a DNA sequence that is not divisible by three. Due to the triplet nature of gene expression by codons, the insertion or deletion can change the reading frame (the grouping of the codons), resulting in a completely different translation from the original. The earlier in the sequence the deletion or insertion occurs, the more altered the protein. A frameshift mutation is not the same as a single-nucleotide polymorphism in which a nucleotide is replaced, rather than inserted or deleted. A frameshift mutation will in general cause the reading of the codons after the mutation to code for different amino acids. The frameshift mutation will also alter the first stop codon (“UAA”, “UGA” or “UAG”) encountered in the sequence. The polypeptide being created could be abnormally short or abnormally long, and will most likely not be functional.

For “single-nucleotide polymorphism” is to be understood as DNA sequence variation occurring when a Single Nucleotide—A, T, C or G—in the genome (or other shared sequence) differs between members of a biological species or paired chromosomes.

A “non-synonymous mutations” or “non-synonymous substitution” is a nucleotide mutation that alters the amino acid sequence of a protein. It is contrasted with synonymous substitutions which do not alter amino acid sequences. As non-synonymous substitutions result in a biological change in the organism, they are subject to natural selection.

When in the present invention the expression “In vitro” is used it encompasses any method modality or use, in which an study is conducted using components of an organism that have been isolated (isolated samples) from their usual biological surroundings in order to permit a more detailed or more convenient analysis than can be done with whole organisms. An “isolated sample” relates according to the invention to any sample (specimen) obtained from an individual and from which a DNA analysis can be performed. Examples of samples include plasma, urine, serum, saliva, semen, etc., as well as in-silico data contained in a computer-readable support (CD, diskettes, MP3).

In the present description the “innate immunity profile”, relates to the gene polymorphisms in Toll-like receptors (TLRs) that are pattern recognition receptors (PRRs) considered to be the primary sensors of pathogens in innate immunity, being able to face pathogens in a first immunity response, much before the adaptive immunity response (that implying T and B lymphocytes) is properly activated.

A mutation may be indicated making reference to the alteration of the gene (nucleotide) sequence, or at protein level, which means that it is indicated the change in the amino acid sequence occasioned due to the nucleotide change in the gene. The nucleotide change may be a deletion of one or more nucleotides, an insertion of one or more nucleotides, or a change of a nucleotide for another with a different base (substitution). When in the present invention the mutation is indicated making reference to the gene alteration, if the alteration is an indel (insertion-deletion), it is indicated by “-”. If the mutation is a change of a nucleotide, it is indicated using the alternating bases, such as G/T (a change of a guanine by a thymine), T/G, T/C, etc., and also the nucleotide position in the corresponding chromosome where the change takes place, such as G/T 3:73542337, which means that the change is in chromosome 3 of Canis lupus familiaris, at the nucleotide position 73542337 according to SNP data base from NCBI http://www.ncbi.nlm.nih.gov/snp/ for the canine chromosomes (equivalent data is retrievable from the database ENSEMBL CanFam3.1—September 2012; http://www.ensembl.org). If the mutation implies a change in the amino acid sequence, the mutation can also be expressed indicating the two amino acids which have been changed in a particular position of the polypeptide sequence, such as S281A of SEQ ID NO: 1, which means that at position 281 of SEQ ID NO: 1 (for example) a serine has been substituted by an alanine. Mutations referred in the present invention are preferably disclosed using the amino acid exchange that takes place in the final protein (i.e. in TLR 1-10) codified for a tlr gene (tlr 1-10). A change of an amino acid by another may be caused by several base pair mutations in the corresponding nucleotide sequence. Any of the degenerate codons for an amino acid causing the amino acid change have to be considered as encompassed in the tlr (1-10) gene fragments of the arrays. As an example, a change of a (S) by a leucine (L) at position 516 of canine TLR2 polypeptide of SEQ ID NO: 1 can take place if the codon for a serine at this position of the open reading frame (for transcribing and translating) is changed due to the mutation of one or more bases of said codon to finally represent a leucine in the same open reading frame. Codon degeneracy and codon tables are well known by the skilled man in the art.

When referring to the gene sequence (nucleotide sequences) the invention uses cursive letters (tlr 1-10 gene). When referring to the amino acid sequences of the proteins codified by said canine genes, capital letters are used (TLR 1-10 protein). If not indicated the contrary, all tlr 1-10 genes and fragments thereof, and all the TLR 1-10 proteins are from Canis genera, and in particular from Canis lupus specie.

For “distinction among individuals” is to be understood that discrimination of isolated samples of different animals of the same breed can be carried out.

Present invention relates to DNA arrays comprising oligonucleotides (i.e. probes) capable of hybridizing with the canine tlr genes or fragments of an isolated sample, said genes including mutations leading to the following changes in the codified protein sequences: S516L in SEQ ID NO: 1, F888C in SEQ ID NO: 2, L457F in SEQ ID NO: 3; and V157A in SEQ ID NO: 4. Thus, the arrays comprise also tlr gene fragments including the above mentioned mutations. As above exposed the oligonucleotides or probes hybridize with tlr genes or fragments of an isolated sample, preferably at a temperature from 50° C. to 70° C., more preferably 60° C. as for a standard TaqMan® type probe. Obviously, other similar conditions may apply depending of the technology used to detect if hybridization took place. The skilled man will know how to adapt the hybridization conditions.

With the detection of at least one of these mutations it can be concluded that the individual will have probably an impaired innate immunity response, or will be susceptible to suffer from any disease related with dysfunctions of the innate immunity system, because at least one of the tlr genes is codifying for an altered protein (TLR).

SEQ ID NOs: 1, 2, 3 and 4 correspond to the protein sequences of the Ensembl database CanFam3.1, release 75 at filing date and being the version of February 2014. In particular,

SEQ ID NO: 1 corresponds with the identification number Ensembl Protein ID ENSCAFP00000012269.2, codified by gene Ensembl Gene ID ENSCAFG00000008351, Ensembl version 2;

SEQ ID NO: 2 corresponds with the identification number Ensembl Protein ENSCAFP00000016726.3, codified by gene Ensembl Gene ID ENSCAFG00000011368, Ensembl version 3;

SEQ ID NO: 3 corresponds with the identification number Ensembl Protein ID ENSCAFP00000023836.3 codified by gene Ensembl Gene ID ENSCAFG00000016172, Ensembl version 3; and

SEQ ID NO: 4 corresponds with the identification number Ensembl Protein ID ENSCAFP00000031505.3, codified by gene Ensembl Gene ID ENSCAFG00000023498, Ensembl version 3.

All genes ID are also entries of CanFam3.1 database, release 75 at filing date and being the version of February 2014.

In a particular embodiment, the canine tlr gene fragments of i) to iv) comprise, respectively, the nucleotide sequences SEQ ID NO: 11 to 13, wherein SEQ ID NO: 11 in the entire nucleotide sequence of tlr 2 gene codifies for the mutation S516L in SEQ ID NO:1; SEQ ID NO: 12 in the entire nucleotide sequence of tlr 6 gene codifies for the mutation L457 in SEQ ID NO: 3; SEQ ID NO: 13 in the entire nucleotide sequence of tlr 8 gene codifies for the mutation V157A in SEQ ID NO: 4; and SEQ ID NO: 47 in the entire nucleotide sequence of tlr 5 gene codifies for the mutation F888C in SEQ ID NO: 2.

The fact that a nucleotide sequence codifies for a mutation, means as above exposed that this tlr gene fragment would give raise to the mutated protein, if forming part of the whole gene. These sequences of the DNA arrays are capable of hybridizing with the complementary chains from a DNA sample isolated from a subject (either with the codifying or the complementary chain including the tlr gene in question)

These tlr gene fragments are oligonucleotides comprising any of SEQ ID NO: 11 to 13, and 47, in particular from 10 to 30 nucleotides, which can hybridize with canine toll-like receptor gene fragments derived or obtained from an isolated sample of a subject (dog or wolf). Indeed, the tlr gene fragments of the array may act as DNA probes (i.e. TaqMan® probes).

In a particular embodiment, optionally in combination with any embodiment above or below, the tlr gene fragments in the array have a length comprised from 10 to 20 nucleotides. In a preferred embodiment the tlr gene fragments in the array have a length selected from 10, 11, 12, 13, 14, 15, 16, 17, 18, 19, and 20 nucleotides.

These gene fragments may be disposed in the array in separated spots or areas (one area for a particular probe), wherein the gene fragments can be in lyophilized form ready to be suspended with any reaction buffer (for example a PCR buffer) in order to promote hybridization with a canine tlr gene fragments of a sample.

In another particular embodiment, optionally in combination with any embodiment above or below, the DNA array according the invention further comprises primers for amplifying toll-like receptor genes, or fragments thereof, codifying for any of the mutations or for the wild-type tlr genes. In a more particular embodiment, the primers have a length from 12 to 40 nucleotides. This particular embodiments relate to arrays further comprising in separate spots the combination of primers/probe for amplifying in an isolated sample the canine tlr gene fragment, in which the particular mutation is desired to be assayed, detected or verified. Examples of commercial arrays including primers and probes for the detection of other genes are those of the type TaqMan Open Array® of Life Technologies.

In a particular embodiment the tlr gene fragments comprising, respectively, any of SEQ ID NO: 11 to 13 and 47, are thus conceived as DNA Taqman® probes, thus comprising a fluorophore at the 5′-end and a quencher at the 3′-end. In a particular embodiment, the fluorophore is selected from 6-carboxyfluorescein (FAM), tetrachlorofluorescein, and VIC; and the quencher is selected from tetramethylrhodamine and dihydrocyclopyrroloindole tripeptide minor groove binder. The array may also comprise primers and other reagents (nucleotides, Taq polymerase, and buffer) for the amplification of the DNA of an isolated sample. VIC is a fluorescent dye that was originally developed by Applied Biosystems, but is now proprietary to Life Technologies. VIC has an absorbance maximum of 538 nm and an emission maximum of 554 nm, thus emitting in the green-yellow part of the visible spectrum. It is used to fluorescently label oligonucleotides at the 5′-end (for use as probes in a variety of real-time PCR, hybridization, and fluorescence-based genetic analysis applications) and is currently only available from LifeTechnologies. VIC's chemical structure is currently not publicly available.

In another particular embodiment the tlr gene fragments of i) to iv) of the first aspect of the invention consist, respectively, in SEQ IDs NO: 11 to 13 and 47, each sequence in the entire corresponding tlr gene codifying for a TLR amino acid sequence with the above indicated mutations.

Besides the mutations indicated above, in a particular embodiment in combination with any of the embodiments above or below, the array further comprises one or more canine tlr gene fragment selected from the group consisting of:
v) A tlr1 gene fragment that in the entire nucleotide sequence of the gene codifies for a change of a serine (S) by an alanine (A) at position 281, and/or for a change of a valine (V) by an isoleucine (I) at position 312 of TLR1 polypeptide of SEQ ID NO: 5;
(vi) A tlr 2 gene fragment that in the entire nucleotide sequence of the gene codifies for a change of a threonine (T) by a methionine (M) at position 606 of canine TLR2 polypeptide of SEQ ID NO: 1
(vii) A trl 3 gene fragment that in the entire nucleotide sequence of the gene codifies for a change of a glutamic (E) by a glutamic (D) at position 176 of canine TLR3 polypeptide of SEQ ID NO: 6;
(viii) A tlr 4 gene fragment that in the entire nucleotide sequence of the gene codifies for a change of a valine (V) by a methionine (M) at position 82, and/or a change of an alanine (A) by a threonine (T) at position 347, and/or a change of a threonine (T) by an alanine (A) at position 577 of canine TLR4 polypeptide of SEQ ID NO: 7;
(ix) A tlr 5 gene fragment that in the entire nucleotide sequence of the gene codifies for a change of a change of a glutamic (E) by an aspartic (D) at position 169, and/or a change of a serine (S) by an arginine (N) at position 177, and/or a change of a valine (V) by a leucine (L) at position 296, and/or a change of a leucine (L) by a serine (S) at position 383, and/or a change of an arginine (R) by a glutamine (Q) at position 402, and/or a change of an arginine (R) by a glutamine (Q) at position 416, and/or a change of a valine (V) by an isoleucine (I) at position 296, and/or a change of a glycine (G) by a serine (S) at position 533, and/or a change of an arginine (R) by a glutamine (Q) at position 734, and/or a change of a glutamic (D) by a tyrosine (Y) 767, and/or a change of a asparagine by a lysine (K) at position 833, and/or a change of an arginie (R) by a cysteine (C) at position 844, and/or the presence of a leucine (L) instead of a serine (S) at position 850, and/or a change of an alanine by a threonine (T) at position 896, and/or a change of an hystidine (H) by a tyrosine (Y) at position 1017, and/or a change of a glycine (G) by a serine (S) at position 1020, and/or a change of an arginine (R) by a glutamine (Q) at position 1049, and/or a change of an alanine (A) by a threonine (T) at position 1078 of canine TLR5 polypeptide of SEQ ID NO: 2);
(x) A tlr 6 gene fragment that in the entire nucleotide sequence of the gene codifies for a change of a tyrosine (Y) by a cysteine (C) at position 182 of canine TLR6 polypeptide of SEQ ID NO: 3;
(xi) A tlr 10 gene fragment that in the entire nucleotide sequence of the gene codifies for a change of a methionine (M) by a valine (V) at position 592 of canine TLR10 polypeptide of SEQ ID NO: 8;
(xii) A tlr 7 gene fragment that in the entire nucleotide sequence of the gene codifies for a change of an alanine by a glycine at position 16, and/or a change of a phenylalanine (F) by a leucine (L) at position 167, and/or a change of a proline (P) by a leucine (L) at position 1066 of canine TLR7 polypeptide of SEQ ID NO: 9;
(xiii) A tlr 8 gene fragment that in the entire nucleotide sequence of the gene codifies for a change of an arginine (R) by glutamine (Q) at position 298 of canine TLR8 polypeptide of SEQ ID NO: 4;
(xiv) A tlr 9 gene fragment that in the entire nucleotide sequence of the gene codifies for a change of a valine (V) by an isoleucine (I) at position 87, and/or a change of a lysine (K) by a glutamic (E) at position 381, and/or a change of a proline (P) by a threonine (T) at position 459, and/or a proline (P) by a leucine (L) at position 787, and/or an arginine (R) by a tryptophan (W) at position 862 of canine TLR 9 polypeptide of SEQ ID NO: 10, and combinations of said gene fragments

For combination of gene fragments is to be understood a combination of two of the gene fragments in the array, three of them, four, five, and until a number including so many gene fragments in such a way that at least one gene fragment codifying in the entire gene for the each one of mutations indicated from (v) to (xiv) is present in the array. In the same way, the expression “one or more” indicates that the array may include combinations of different tlr gene fragments from tlr 1 to tlr 10, as well as different gene fragments of the same tlr gene (i.e., two tlr 9 gene fragments, both codifying in the entire tlr 9 gene for a TLR9 amino acid sequence including different mutations).

SEQ ID NOs: 5, 6, 7, 8, 9 and 10 correspond to the protein sequences of the Ensembl database CanFam3.1, release 75 at filing date and being the version of February 2014. In particular, SEQ ID NO: 5 corresponds with the identification number Ensembl Protein ID ENSCAFP00000032660.1, codified by gene Ensembl Gene ID ENSCAFG00000024010, version 1; SEQ ID NO: 6 corresponds with the identification number Ensembl Protein ID ENSCAFP00000011004.3, codified by gene Ensembl Gene ID ENSCAFG00000007406, version 3; SEQ ID NO: 7 corresponds with the identification number Ensembl Protein ID ENSCAFP00000031395.2, codified by gene Ensembl Gene ID ENSCAFG00000003518, version 3; SEQ ID NO: 8 corresponds with the identification number Ensembl Protein ID ENSCAFP00000023840.1, codified by gene Ensembl Gene ID ENSCAFG00000016175, version 1; SEQ ID NO: 9 corresponds with the identification number Ensembl Protein ID ENSCAFP00000017193.3, codified by gene Ensembl Gene ID ENSCAFG00000011698, version 3, and SEQ ID NO: 10 corresponds with the identification number Ensembl Protein ID ENSCAFP00000030804.2, codified by gene Ensembl Gene ID ENSCAFG00000023201, version 2. All genes ID are also entries of CanFam3.1 database, release 75 at filing date and being the version of February 2014.

In a particular embodiment, optionally in combination with any embodiments below or above, the DNA array comprises canine tlr gene fragments, wherein the tlr 1 gene fragment of (v) is selected from a group consisting of a sequence comprising SEQ ID NO: 14, a sequence comprising SEQ ID NO: 15, and combinations thereof; the tlr 2 gene fragment of (vi) is a sequence comprising SEQ ID NO: 16; the tlr 3 gene fragment of (vii) is a sequence comprising SEQ ID NO: 17; the tlr 4 gene fragment of (viii) is selected from a group consisting of a sequence comprising SEQ ID NO: 18, a sequence comprising SEQ ID NO: 19, a sequence comprising SEQ ID NO: 20, and combinations thereof; the tlr 5 gene fragment of (ix) is selected from a group consisting of a sequence comprising any of sequences SEQ ID NO: 21-35 and SEQ ID NO: 52, and combinations thereof; the tlr 6 gene fragment of (x) is a sequence comprising SEQ ID NO: 36; the tlr 10 gene fragment of (xi) is a sequence comprising SEQ ID NO: 46; the tlr 7 gene fragment of (xii) is selected from a group consisting of a sequence comprising any of sequences SEQ ID NO: 37-39, and combinations thereof; the tlr 8 gene fragment of (xiii) is a sequence comprising SEQ ID NO: 40; and the tlr 9 gene fragment of (xiv) is selected from a group consisting of a sequence comprising any of sequences 41-45, and combinations thereof.

SEQ ID NO: 14 (CATTTGAAATTGAGAAATAC) codifies in the entire tlr 1 gene fragment for the mutation S281A in SEQ ID NO: 5; SEQ ID NO: 15 (ACACCAAGTCGTTAGTAAT) codifies in the entire tlr 1 gene fragment for the mutation V312I in SEQ ID NO: 5.

SEQ ID NO: 16 (CCCGCCGTGAGCAG) codifies in the entire tlr 2 gene fragment for the mutation T606M in SEQ ID NO: 1.

SEQ ID NO: 17 (TCTCCAAGAGCTTCTG) codifies in the entire tlr 3 gene fragment for the mutation E176D in SEQ ID NO: 6.

SEQ ID NO: 18 (CAAAACTGCAGGTGCTGG) codifies in the entire tlr 4 gene fragment for the mutation V82M in SEQ ID NO: 7; SEQ ID NO: 19 (CCCATGCGGGAAAT) codifies in the entire tlr 4 gene fragment for the mutation A347T in SEQ ID NO: 7; SEQ ID NO: 20 (AAGTTATTCCGAGTAAGATT) codifies in the entire tlr 4 gene fragment for the mutation T577A in SEQ ID NO: 7.

SEQ ID NO: 21 (TTCCCCGAAGCCCTG) codifies in the entire tlr 5 gene fragment for the mutation E169D in SEQ ID NO: 2; SEQ ID NO: 22 (TGTTTGCCGTCACTCAA) codifies in the entire tlr 5 gene fragment for the mutation S177N in SEQ ID NO: 2; SEQ ID NO: 23 (TCCGGGCCGTCACC) codifies in the entire tlr 5 gene fragment for the mutation V296I in SEQ ID NO: 2; SEQ ID NO: 24 (AGGCGCAACAAAG) codifies in the entire tlr 5 gene fragment for the mutation L383S in SEQ ID NO: 2; SEQ ID NO: 25 (TCCTTCCGGGAGCTG) codifies in the entire tlr 5 gene fragment for the mutation R402Q in SEQ ID NO: 2; SEQ ID NO: 26 (CTCAACCGGATCCC) codifies in the entire tlr 5 gene fragment for the mutation R416Q in SEQ ID NO: 2; SEQ ID NO: 27 (TCGCACGGCTTCG) codifies in the entire tlr 5 gene fragment for the mutation G533S in SEQ ID NO: 2; SEQ ID NO: 28 (AGCCCCCGGAACAC) codifies in the entire tlr 5 gene fragment for the mutation R734Q in SEQ ID NO: 2; SEQ ID NO: 29 (TCAGGTCGAGGCCC) codifies in the entire tlr 5 gene fragment for the mutation D767Y in SEQ ID NO: 2; SEQ ID NO: 30 (CGCAGCGCACGTC) codifies in the entire tlr 5 gene fragment for the mutation R844C in SEQ ID NO: 2; SEQ ID NO: 52 (CCCAGCTTGCTTGC) codifies in the entire tlr 5 gene fragment for the mutation L850S in SEQ ID NO: 2; SEQ ID NO: 31 (CTCGTGGCCGCCAAG) codifies in the entire tlr 5 gene fragment for the mutation A896T in SEQ ID NO: 2; SEQ ID NO: 32 (CCAGGTGGGACGCG) codifies in the entire tlr 5 gene fragment for the mutation H1017Y in SEQ ID NO: 2; SEQ ID NO: 33 (CCTGGACGGCGCCCT) codifies in the entire tlr 5 gene fragment for the mutation G1020S in SEQ ID NO: 2; SEQ ID NO: 34 (CAGCGCCGGTACTT) codifies in the entire tlr 5 gene fragment for the mutation R1049Q in SEQ ID NO: 2; SEQ ID NO: 35 (CGCGCGCCCCTC) codifies in the entire tlr 5 gene fragment for the mutation A1078T in SEQ ID NO: 2.

SEQ ID NO: 36 (TTCTTTTGCATAATAACC) codifies in the entire tlr 6 gene fragment for the mutation Y182C in SEQ ID NO: 3.

SEQ ID NO: 37 (AGGGAGCCTGCTTCT) codifies in the entire tlr 7 gene fragment for the mutation A16G in SEQ ID NO: 9; SEQ ID NO: 38 (TGCATCGGAAATCG) codifies in the entire tlr 7 gene fragment for the mutation F167L in SEQ ID NO: 9; SEQ ID NO: 39 (CTTGAGAAGCCCCTTCAG) codifies in the entire tlr 7 gene fragment for the mutation P1066L in SEQ ID NO: 9.

SEQ ID NO: 40 (CCCTCCGGAAGATT) codifies in the entire tlr 8 gene fragment for the mutation R298Q in SEQ ID NO: 4.

SEQ ID NO: 41 (TTGTCCACTTCGTCCACC) codifies in the entire tlr 9 gene fragment for the mutation V87I in SEQ ID NO: 10; SEQ ID NO: 42 (CGCTCAGCAAGACCA) codifies in the entire tlr 9 gene fragment for the mutation K381E in SEQ ID NO: 10; SEQ ID NO: 43 (CCCAGTGGGCCCAGAG) codifies in the entire tlr 9 gene fragment for the mutation P459T in SEQ ID NO: 10; SEQ ID NO: 44 (ACGGCTAGGCAGGCC) codifies in the entire tlr 9 gene fragment for the mutation P787L in SEQ ID NO: 10; SEQ ID NO: 45 (CCCGCCGCCGCC) codifies in the entire tlr 9 gene fragment for the mutation R862W in SEQ ID NO: 10.

SEQ ID NO: 46 (CCACAGCCATCCCCAG) codifies in the entire tlr 10 gene fragment for the mutation M592V in SEQ ID NO: 8.

In another particular embodiment, optionally in combination with any of the embodiments below or above, the canine tlr gene fragments of v) to xiv) of the first aspect of the invention consist, respectively, in SEQ IDs NO: 14 to 46 and SEQ ID NO: 52, each sequence in the entire corresponding tlr gene codifying for a TLR amino acid sequence with the above indicated mutations

As above indicated for SEQ ID NO: 11-13 and 47, any of SEQ ID NO: 14 to 46 and 52, or a tlr gene fragment comprising any of them, may act as DNA probes (i.e. TaqMan probes). In a particular case, the tlr gene fragments comprising or consisting in any of SEQ ID NO: 14 to 46 and SEQ ID NO: 52 are conceived as DNA Taqman probes, thus comprising a fluorophore at the 5′-end and a quencher at the 3′-end. The array may also comprise primers and other reagents (nucleotides, Taq polymerase, and buffer) for the amplification of the DNA of an isolated sample. They may be disposed in the array in separated spots or areas (one area for a particular probe), wherein the gene fragments can be in lyophilized form ready to be suspended with any reaction buffer (for example a PCR buffer) in order to promote hybridization with a canine TLR gene fragment of a sample.

Thus, in those particular embodiments where also mutations indicated from (v) to (xiv) are to be detected, the tlr gene fragments in the array have a length comprised from 10 to 30 nucleotides, more in particular from 10 to 20 nucleotides. Most particularly a length selected from 10, 11, 12, 13, 14, 15, 16, 17, 18, 19, and 20 nucleotides.

With the new mutations herewith identified, relevant mutations of canine tlr genes are detected in an isolated sample. All these mutations in the different genes give raise to changes in the amino acid sequence of the proteins (TLR) codified by said genes. As will be detailed in the examples below, most of these mutations lead to deleterious proteins and, thus, they result damaging in the sense that the protein will probably lose its function. Loose of function of a protein is finally translated to an impaired whole functionality of the pathway wherein it is involved. In the present case, these non-functionalities of the proteins defining the TLR lead to innate immunity diseases, such as infectious and autoimmune diseases including, but not limited to chronic enteropathies, osteoarthritis, endometrium infections, sino-nasal aspergillosis, idiopathic lymphoplasmacytic rhinitis, Leishmania infections, atopic dermatitis, inflammatory bowel syndrome (IBS), inflammatory bowel disease (IBD), Chron's disease, viral infections and bacterial infections.

Complementation of the array may be made by adding other probes (oligonucleotides) capable of detecting other mutations in the canine tlr genes. Examples of these probes are capable of detecting the SNPs selected from the group consisting of: rs23585044, rs23572381, rs23572380, rs22410121, rs8958543, rs22120766, rs22157966, rs22145736, rs22189454, rs22189456, rs22124023, rs22123995, rs24029590, rs9070448, rs9070450, rs9070451, rs9070452, rs9070447, rs9125247, rs24029975, rs23570247, rs24607342, rs24607358, rs9188882, rs22882109, and rs23518574, and combinations of said single nucleotide polymorphisms. All these references are retrievable from database dbSNP “Short Genetic Variations” from the National Center for Biotechnology Information (NCBI) http://www.ncbi.nlm.nih.gov/snp/.

In another particular embodiment, optionally in combination with an embodiment above or below, the DNA array comprises oligonucleotides capable of detecting wild-type canine tlr gene fragments. Thus, in a particular embodiment of the array, it further comprises tlr gene fragments codifying for the wild-type tlr gene in a particular possibly mutated point. These oligonucleotides (i.e. wild-type gene fragments) allow also determining if the test sample comprises wild-type forms of the genes, which are present in heterozygote individuals for said feature or character (i.e. a particular TLR). Alternatively, in case of a homozygote individual, these probes for detecting non-mutated tlr genes fragments represent a positive control for the array.

In a particular embodiment, the array comprises wild-type tlr gene fragments for each of the tlr gene fragments codifying for a specified mutation.

In a particular embodiment, optionally in combination with any embodiments below or above, the DNA array comprises the following pairs of primers (forward and reverse) or pairs of sequences including them usable as primers for elongating the canine tlr genes including the points with the possible mutations:

For canine TLR1, SEQ ID NO: 53 (AGTTGGTTTGGCATACAAGCATAGA) and SEQ ID NO: 54 (TCTGAAGTCAGGGTAACCTTGTAGT) for amplifying wild-type and mutated TLR1 gene fragment codifying for the mutation S281A in SEQ ID NO: 5; and SEQ ID NO: 55 (GACACTTCACTGAAGGCCTTATCT) and SEQ ID NO: 56 (AGATTTTATAGATATAACTTTGTGGCAAATTGAATGC) for amplifying wild-type and mutated TLR1 gene fragment codifying for the mutation V312I in SEQ ID NO: 5.

For canine TLR2, SEQ ID NO: 57 (CCACCTTACAAATTATGAGAATCAGCAGAA) and SEQ ID NO: 58 (AGCCTGTGAAAGGAATCCAGTTG) for amplifying wild-type and mutated TLR2 gene fragment codifying for the mutation S516L in SEQ ID NO: 1; and SEQ ID NO: 59 (GCCGTGTGCTGTGTCTTG) and SEQ ID NO: 60 (ACAGCCCGTGGAAATGGT) for amplifying wild-type and mutated TLR2 gene fragment codifying for the mutation T606M in SEQ ID NO: 1.

For canine TLR3, SEQ ID NO: 61 (AAGTTCTTCACGCCTCAGTACATT) and SEQ ID NO: 62 (ATTAGGAAGTCAGCTCCAATTGGAAAA) for amplifying wild-type and mutated TLR3 gene fragment codifying for the mutation E176D in SEQ ID NO: 6.

For canine TLR4, SEQ ID NO: 63 (CAGCCATTGCTTCTCCAACTTC) and SEQ ID NO: 64 (CTTGGGCAATGTGTAAAAGCTCAT) for amplifying wild-type and mutated TLR4 gene fragment codifying for the mutation V82M in SEQ ID NO: 7; SEQ ID NO: 65 (AGATGGCAACGGTTGGAAATAGTTA) and SEQ ID NO: 66 (GGAAGTGAGAACAAACTCCTTGAGA) for amplifying wild-type and mutated TLR4 gene fragment codifying for the mutation A347T in SEQ ID NO: 7; and SEQ ID NO: 67 (GAACAAGGACAACAGCATTTTCCAA) and SEQ ID NO: 68 (TCTGATGTTCACAGTCACAAGCA) for amplifying wild-type and mutated TLR4 gene fragment codifying for the mutation T577A in SEQ ID NO: 7.

For canine TLR5, SEQ ID NO: 69 (CACGTTCTCCCTCTTCATCTTCTC) and SEQ ID NO: 70 (CAAAGGCCCCGGAGCTT) for amplifying wild-type and mutated TLR5 gene fragment codifying for the mutation F888C in SEQ ID NO: 2; SEQ ID NO: 71 (GTATCCTCCGCCTGCCATT) and SEQ ID NO: 72 (TTACCTTGAGGAGAGTCTGTTTGC) for amplifying wild-type and mutated TLR5 gene fragment codifying for the mutation E169D in SEQ ID NO: 2; SEQ ID NO: 73 (CTCCGCCTGCCATTTTCC) and SEQ ID NO: 74 (GGTCCTTGAGAAACACTTACCTTGA) for amplifying wild-type and mutated TLR5 gene fragment codifying for the mutation S177N in SEQ ID NO: 2; SEQ ID NO: 75 (CTCCTGCTGAGCTTCAACTACA) and SEQ ID NO: 76 (GCAGCCGCTCCAGGA) for amplifying wild-type and mutated TLR5 gene fragment codifying for the mutation V296I in SEQ ID NO: 2; SEQ ID NO: 77 (CGTGTTGACAGACGGTTATTTCAGA) and SEQ ID NO: 78 (CTCGAGGCTCCCAATCTGATTTT) for amplifying wild-type and mutated TLR5 gene fragment codifying for the mutation L383S in SEQ ID NO: 2; SEQ ID NO: 79 (GGGAGCCTCGAGCTTCAC) and SEQ ID NO: 80 (ACACGCAGCCGGGATC) for amplifying wild-type and mutated TLR5 gene fragment codifying for the mutation R402Q in SEQ ID NO: 2; SEQ ID NO: 81 (GCTGGGTTCCCTGAGGTC) and SEQ ID NO: 82 (AGGGAGAGCGCCTTGC) for amplifying wild-type and mutated TLR5 gene fragment codifying for the mutation R416Q in SEQ ID NO: 2; SEQ ID NO: 83 (TGCTGCGGCTGGATCTG) and SEQ ID NO: 84 (GGGCGTTCAGGGAGAAGA) for amplifying wild-type and mutated TLR5 gene fragment codifying for the mutation G533S in SEQ ID NO: 2; SEQ ID NO: 85 (GGGCGGTGCTGGGA) and SEQ ID NO: 86 (TGTGGTTCAGGTGCAGCA) for amplifying wild-type and mutated TLR5 gene fragment codifying for the mutation R734Q in SEQ ID NO: 2; SEQ ID NO: 87 (GACCTCACGGCGCTGA) and SEQ ID NO: 88 (GCGTGCTCAGCCTGTTG) for amplifying wild-type and mutated TLR5 gene fragment codifying for the mutation D767Y in SEQ ID NO: 2; SEQ ID NO: 89 (GTGGCTCAACCGGACCAA) and SEQ ID NO: 90 (AGCCCTCCATGGAGACAGA) for amplifying wild-type and mutated TLR5 gene fragment codifying for the mutation R844C in SEQ ID NO: 2; SEQ ID NO: 105 (TCCCGCGCAGACGTG) and SEQ ID NO: 106 (AGCCCTCCATGGAGACAGA) for amplifying wild-type and mutated TLR5 gene fragment codifying for the mutation L850S in SEQ ID NO: 2; SEQ ID NO: 91 (CCTCTTCATCTTCTCCACCGT) and SEQ ID NO: 92 (GCCTTGTAACAGAGGAAGCAAAG) for amplifying wild-type and mutated TLR5 gene fragment codifying for the mutation A896T in SEQ ID NO: 2; SEQ ID NO: 93 (TGCCTGGAGGCCTTCG) and SEQ ID NO: 94 (CACGACCACCAGGACGAG) for amplifying wild-type and mutated TLR5 gene fragment codifying for the mutation H1017Y in SEQ ID NO: 2; SEQ ID NO: 95 (TGCCTGGAGGCCTTCG) and SEQ ID NO:96 (CCCACGACCACCAGGAC) for amplifying wild-type and mutated TLR5 gene fragment codifying for the mutation G1020S in SEQ ID NO: 2; SEQ ID NO: 97 (AGGCACCCGGCCATC) and SEQ ID NO: 98 (CCACGTCCTGCAGATCCT) for amplifying wild-type and mutated TLR5 gene fragment codifying for the mutation R1049Q in SEQ ID NO: 2; and the pair SEQ ID NO: 99 (ACGCTCTCCCGACACATC) and SEQ ID NO: 100 (CGCAGCGGGATGCC) for amplifying wild-type and mutated TLR5 gene fragment codifying for the mutation A1078T.

For canine TLR6, SEQ ID NO: 101 (GTTTTCAGATGTTTACCTCCCAAGGT) and SEQ ID NO: 102 (AGCTTCATGATTGGTTTAGGAATGCT) for amplifying wild-type and mutated TLR6 gene fragment codifying for the mutation L457F in SEQ ID NO: 3; and the pair of primers SEQ ID NO: 103 (ATCTGCTACCAATTGCTCATTT) and SEQ ID NO: 104 (AGTGTTTTTGTATCTAGAATTTGAAGACTTCCT) for amplifying wild-type and mutated TLR6 gene fragment codifying for the mutation Y182C in SEQ ID NO: 3.

For canine TLR7, SEQ ID NO: 107 (AGAGAGAGGCAGGGACACA) and SEQ ID NO: 108 (GGATCGAGTCCCACATCGG) for amplifying wild-type and mutated TLR7 gene fragment codifying for the mutation A16G in SEQ ID NO: 9; SEQ ID NO: 109 (CCAGCTGGACTATCTGGTAGAGAT) and SEQ ID NO: 110 (CCCCAGTCGAACAGGTATACAG) for amplifying wild-type and mutated TLR7 gene fragment codifying for the mutation F167L in SEQ ID NO: 9; and SEQ ID NO: 111 (TCATGGATGAAAAAGTGGACGTCAT) and SEQ ID NO: 112 (GGAGCTGGAGGAACTTGGATTT) for amplifying wild-type and mutated TLR7 gene fragment codifying for the mutation P1066L in SEQ ID NO: 9.

For TLR8, SEQ ID NO: 113 (GGTCTTTGAAGGAACTTAGCCTGAT) and SEQ ID NO: 114 (CGTGAGCCCAGACGTATTCTTT) for amplifying wild-type and mutated TLR8 gene fragment codifying for the mutation V157A in SEQ ID NO: 4; and SEQ ID NO: 115 (GCTTCGCTACCTAAACCTCTCTAG) and SEQ ID NO: 116 (CATGTTGTCAAACCACGTTGCA) for amplifying wild-type and mutated TLR8 gene fragment codifying for the mutation R298Q in SEQ ID NO: 4.

For canine TLR9, SEQ ID NO: 117 (CCACCACCTCCATGACTATGACT) and SEQ ID NO: 118 (GGCAGTTCCACTTGAGATTGAGA) for amplifying wild-type and mutated TLR9 gene fragment codifying for the mutation V87I in SEQ ID NO: 10; SEQ ID NO: 119 (GCTGGACATACATGGCATCTTCTT) and SEQ ID NO: 120 (GGTGGGCCAGCGACT) for amplifying wild-type and mutated TLR9 gene fragment codifying for the mutation K381E; SEQ ID NO: 121 (GGAGAGAGTCTGGCCACAGT) and SEQ ID NO: 122 (CGGCATGAAGGCCTCTGA) for amplifying wild-type and mutated TLR9 gene fragment codifying for the mutation P459T in SEQ ID NO: 10; SEQ ID NO: 123 (GCTGGAGGTGCAGGCT) and SEQ ID NO: 124 (GGCTGCCGCACTTGAC) for amplifying wild-type and mutated TLR9 gene fragment codifying for the mutation P787L in SEQ ID NO: 10; and SEQ ID NO: 125 (GTGCCTGGCCTGGCT) and SEQ ID NO: 126 (GCCTTGTCGAAGACCACGAA) for amplifying wild-type and mutated TLR9 gene fragment codifying for the mutation R862W in SEQ ID NO: 10.

For canine TLR9, the pair of primers SEQ ID NO: 127 (TCTGTTGATTGTCACCATTGTGGTT) and SEQ ID NO: 128 (CAGATCAAAGTAGAAGCAGCAGAAG) for amplifying wild-type and mutated TLR10 gene fragment codifying for the mutation M592V in SEQ ID NO: 8.

Each of the above listed pair of primers serve also, as indicated, for amplifying the corresponding wild-type canine tlr gene fragments, said wild-type gene fragments codifying for the peptide fragments of TLR protein sequences including the amino acid that could be mutated but it is not mutated according to the reference sequences. Reference sequences are those identified by the ENSEMBL database accession number above listed.

Another particular embodiment of the array of the invention, it includes wild-type canine tlr gene fragments.

Another aspect of present invention is the peptide sequence comprising SEQ ID NO: 48. In a particular embodiment, the peptide sequence comprising SEQ ID NO: 48, further comprises additionally other mutations, in particular a change of a threonine (T) by a methionine (M) at position 606 of canine TLR2 polypeptide of SEQ ID NO: 1.

In the same way, particular embodiments of the peptide sequences comprising any of SEQ ID NO: 49 to 51, relate to these sequences further comprising other mutations, in particular those mutations identified in embodiments above in the corresponding sequences SEQ ID NO: 1, 2, 3, or 4, such as mutation T606M of SEQ ID NO: 1; mutation Y182C of SEQ ID NO: 3; mutation R298Q of SEQ ID NO: 4; and mutations V296I and/or L383S of SEQ ID NO: 2.

Another aspect of the invention relates to a method for identifying mutations in canine toll-like receptor genes. In a particular embodiment the method comprises:

obtaining an isolated sample of an animal of the Canis genera comprising DNA;

adding said isolated animal sample comprising DNA to a DNA array as defined in any of the aspects and embodiments disclosed above; and

determining if the toll-like receptor gene fragments of the array hybridize with sample gene fragments.

In a particular embodiment of the method for identifying mutations, the isolated DNA sample is added to a DNA array that comprises canine tlr gene fragments as those defined from i) to iv) and comprising, respectively, the nucleotide sequences SEQ ID NO: 11 to 13, wherein SEQ ID NO: 11 (CTCCTTCGAGAAAGC) in the entire nucleotide sequence of tlr 2 gene codifies for the mutation S516L in SEQ ID NO:1; SEQ ID NO: 12 (TTATCGTGAAGATCAAGC) in the entire nucleotide sequence of tlr 6 gene codifies for the mutation L457 in SEQ ID NO: 3; SEQ ID NO: 13 (CATCATCTGGGTAACGAA) in the entire nucleotide sequence of tlr 8 gene codifies for the mutation V157A in SEQ ID NO: 4; and SEQ ID NO: 47 (CCAGGAGGAACAGCGT) in the entire nucleotide sequence of tlr 5 gene codifies for the mutation F888C in SEQ ID NO: 2.

In a more particular embodiment of the method for identifying mutations, the DNA sample is added to an array that further comprises oligonucleotides (i.e. tlr gene fragments) comprising any of the sequences SEQ ID NO: 14 to SEQ ID NO: 46, and SEQ ID NO: 52. In a more particular embodiment, the oligonucleotides in the array consist in any combination of oligonucleotides sequences SEQ ID NO: 14 to SEQ ID NO: 46 and SEQ ID NO: 52.

Another particular embodiment of the method for identifying mutations, optionally in combination with any of the embodiments below or above, the step of determining if the toll-like receptor gene fragments of the array (oligonucleotides of the array) hybridize with sample gene fragments codifying for a mutation, is carried out by a means selected from the group consisting of chemiluminescence, fluorescence, and radioisotope technologies.

Determination of hybridization allows concluding if a particular mutation is present or not in a sample.

In another particular embodiment of the method, optionally in combination with any of the embodiments below or above, the sample comprising DNA of an animal of the Canis genera, relates to a sample wherein DNA is present or that derives or comes from a sample comprising RNA that has been converted to the corresponding cDNA by means of the retro transcription techniques (for example, RT-PCR).

As above exposed, the invention also relates to an in vitro method for the individual identification of a Canis genera animal, comprising determining the presence or absence of canine toll-like receptor mutations in an isolated sample by means of the DNA array as defined above; and establishing a combination of detected and non-detected mutations in said sample. In a particular embodiment of this identification method, the array is the one comprising tlr gene fragments codifying at least for the mutations disclosed in Table 2, and most particularly codifying at least for the mutations disclosed in Table 2 and further for mutation V312I of TLR1 (in SEQ ID NO: 5), and for the mutations V296L, N833K, S850L of TLR5 (in SEQ ID NO: 2). In another particular embodiment, the DNA array comprises oligonucleotide probes from 10 to 30 nucleotides, which are tlr gene fragments capable of identifying toll-like receptor mutations indicated in Table 1 below; and establishing a combination of detected and non-detected mutations in said sample.

As indicated before, this method for individual identification with only the analysis of tlr gene fragments is also for distinguishing among individuals of the Canis genus.

It is thus another aspect of the invention an in vitro method for the distinction among individuals of a Canis genus animal, comprising determining the presence or absence of canine toll-like receptor mutations in an isolated sample by means of the DNA array as defined above and comprising tlr gene fragments codifying at least for the mutations disclosed in Table 2 and further for mutation V312I of TLR1 (in SEQ ID NO: 5), and for the mutations V296L, N833K, S850L of TLR5 (in SEQ ID NO: 2); and establishing a combination of detected and non-detected mutations in said sample. Thus, the array comprises tlr gene fragments codifying at least for forty mutations.

In a more particular embodiment of the in vitro method for the distinction among individuals of a Canis genus animal, the DNA array comprises oligonucleotide probes from 10 to 30 nucleotides, which are tlr gene fragments capable of identifying toll-like receptor mutations indicated in Table 1; and establishing a combination of detected and non-detected mutations in said sample.

In any of the methods and arrays of the invention the sample containing DNA is selected from the group consisting of blood, saliva, faeces, urine, skin, hair, muscle or any other tissue from which DNA can be obtained. That is, in particular from the group consisting of blood, saliva, faeces, urine, hair, skin tissue biopsy, and muscle tissue biopsy.

In a particular embodiment of the method, the DNA sample is a computer readable support containing the sequence of canine tlr genes of an individual, obtained for example by massive sequencing, or from a previous analysis. Then, the sequences are aligned with the sequences defining the oligonucleotides comprised in the array, also in a computer readable support.

As above exposed, the combined determination of the particular mutations gives relevant information regarding a toll-like receptor in question, as well as relevant information regarding innate immunity response, an/or diseases related with an innate immunity dysfunction, such as atopic dermatitis, Inflammaory bowel syndrome (IBS), Inflammatory Bowel disease (IBD), Chron's disease, viral infections and bacterial infections.

Thus, another aspect of the invention is an in vitro method for the analysis of the genetic predisposition of an individual of the Canis genera to suffer from any disease related with dysfunctions of the innate immunity system, the method comprising carrying out the method for identifying mutations in canine toll-like receptor genes as defined above, and correlating any detected mutation with a disease. In a particular embodiment, thus, the method for the analysis of the genetic predisposition of an individual of the Canis genera to suffer from any disease related with dysfunctions of the innate immunity system comprises determining in an isolated sample of an individual if at least the following mutations are present: a change of a serine (S) by a leucine (L) at position 516 of canine TLR2 polypeptide of SEQ ID NO: 1; a change of a leucine (L) by a phenylalanine (F) at position 457 and a change of a proline (P) by a leucine (L) at position 579 of canine TLR6 polypeptide of SEQ ID NO: 3; and a change of a valine (V) by an alanine (A) at position 157 of canine TLR8 polypeptide of SEQ ID NO: 4. In a more particular embodiment, the method comprises determining in an isolated sample of an individual if at least the following mutations are present: a change of a serine (S) by a leucine (L) at position 516 of canine TLR2 polypeptide of SEQ ID NO: 1; a change of a phenylalanine (F) by a cysteine (C) at position 888 of canine TLR5 polypeptide of SEQ ID NO: 2; a change of a leucine (L) by a phenylalanine (F) at position 457 and a change of a proline (P) by a leucine (L) at position 579 of canine TLR6 polypeptide of SEQ ID NO: 3; and a change of a valine (V) by an alanine (A) at position 157 of canine TLR8 polypeptide of SEQ ID NO: 4

Detection of the mutations are carried out using the probes (oligonucleotides, which indeed are canine tlr gene fragments) as defined in SEQ ID NO: 11, 12, 13, and 47. In another particular embodiment, the mutations in canine tlr genes are further detected using any combination of the oligonucleotides comprising or consisting in SEQ ID NO: 14 to SEQ ID NO: 46 and SEQ ID NO: 52.

In a particular embodiment of any of the methods of the invention (for determining mutations, or for the analysis of susceptibility to a disease), as well as of the array to which a DNA sample of an individual will be added, the Canis genera individual is selected from the species Canis lupus and Canis lupus familiaris. In a most preferred embodiment, the individual is from the Canis lupus familiaris species (commonly known as domestic dogs). Among the species Canis lupus familiaris there are many breeds. The methods and arrays of the invention are applicable to samples of all breeds. In the same way, due to the phylogenetically proximity of wolves (Canis lupus), the method and arrays are also applicable to wolves.

Dog breeds are groups of closely related and visibly similar domestic dogs, which are all of the subspecies Canis lupus familiaris, having characteristic traits that are selected and maintained by humans, bred from a known foundation stock.

In another particular embodiment of any of the methods of the invention (for determining mutations, for individual identification or for the analysis of susceptibility to a disease), it further includes the step of further collecting and/or providing and/or saving data derived from previous steps in a data carrier. A data carrier includes means (paper, CD, digitalized information, computer archives, or sound recordings) that contain meaningful information data.

Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. The following examples are provided by way of illustration, and they are not intended to be limiting of the present invention. Furthermore, the present invention covers all possible combinations of particular and preferred embodiments described herein.

### EXAMPLES

Following examples illustrate the arrays of the invention as well as a way of carrying out the methods of the invention in which the presence or absence of relevant mutations in canine TLRs are detected.

### Example 1

### Identification of Relevant Mutations in Canine TLRs Genes

For the performance of the Examples the following materials and methods were used

DNA sources:

Samples available from the DNA bank at the SVGM (Servei Veterinari de Genètica Molecular, Molecular Genetics Veterinary Service, UAB) were used. Total DNA from blood cells had been extracted either as described elsewhere (Francino, O. et al., 2006. Advantages of real-time PCR assay for diagnosis and monitoring of canine leishmaniosis. Veterinary Parasitology 137: 214-221) or using QIAamp DNA Mini Kit (Qiagen).

DNAs from 7 different dog (Canis lupus familiaris) breeds, including Beagle, German shepherd, Yorkshire, French bulldog, Boxer, Labrador and Shar Pei were used. Also 2 different populations of wolves (Canis lupus) were analyzed, Iberian and Russian. Polymorphism analyses were performed using DNA pools, which were constituted of 200 ng of DNA from 25 unrelated dogs (in exception of one pool of French bulldogs, which only 10 individuals were used). Two pools of each breed were analyzed, in exception of Shar Pei (only 1 pool) and Boxer (3 pools). Pools of wolves were constituted of 50 individuals.

Some DNA samples used for a first massive sequencing analyses were also chosen to be individually genotyped in order to validate SNPs in a TaqMan Open Array® designed (see Example below). 15 Beagle, 15 Boxer, 15 French bulldog, 15 Labrador, 14 German shepherd dog, 12 Yorkshire, 11 Shar Pei and 4 wolves were used.

Exon capture and massive sequencing for SNP discovery:

Twenty exonic regions of 10 canine tlr genes annotated in CanFam 2.0 were chosen to perform the analysis. 2372 bp for tlr 1 (Ensembl Gene ID ENSCAFG00000024010), 2357 bp for tlr 2 (Ensembl Gene ID ENSCAFG00000008351), 2717 bp for tlr 3 (Ensembl Gene ID ENSCAFG00000007406), 2522 bp for tlr 4 (Ensembl Gene ID ENSCAFG00000003518),

2576 bp for tlr 5 (Ensembl Gene ID ENSCAFG00000011368), 2413 for tlr 6 (Ensembl Gene ID ENSCAFG00000016172), 4241 bp for tlr7 (Ensembl Gene ID ENSCAFG00000011698), 3119 bp for tlr 8 (Ensembl Gene ID ENSCAFG00000023498), 3015 bp for tlr 9 (Ensembl Gene ID ENSCAFG00000023201) and 2423 bp for tlr 10 (Ensembl Gene ID ENSCAFG00000016175).

All gene identifications proceed from the database Ensembl project, which is a joint project between European Bioinformatics Institute (EBI), an outstation of the European Molecular Biology Laboratory (EMBL), and the Wellcome Trust Sanger Institute (WTSI). Access of the database is available at http://www.ensembl.org.

Oligonucleotides (120 nt) were first automatically designed for the enrichment of selected regions (https://earray.chem.agilent.com/suredesign/). Regions rejected in the automated design, because of the presence of gaps, repeats or shorter sizes than required (at least 120 nucleotides) were manually redesigned. Finally 235 capture oligonucleotides were designed, for Agilent Sure-select library, which covered 28,200 bases. High-throughput sequencing was performed using 2 lanes of Illumina HISEQ, with 8-labelled pools each, at CNAG (Centre Nacional d'Anàlisi Genòmica).

Sequences obtained were mapped to database CanFam 3.1 of Ensembl project (released September 2012). All pools were analysed together for variant calling, for better comparison. Positions with a total coverage of less than 8,000 reads were filtered out. Alternative variant frequencies were estimated for each breed pool and wolf populations. The variants were annotated with statistical information from the Genome Analysis Tool Kit (GATK) and functional annotations were added from Ensembl using snpEff (http://snpeff.sourceforge.net/). Variants already described for dog, human, cow and pig TLRs were retrieved from ENSEMBL (CanFam 3.1).

Prediction of functional impact of non-synonymous SNPs:

The functional impact of non-synonymous mutations detected was predicted using Polyphen-2 (http://genetics.bwh.harvard.edu/pph2/index.shtml) (Adzhubei I a, Schmidt S, Peshkin L, Ramensky V E, Gerasimova A, Bork P, Kondrashov A S, Sunyaev S R: A method and server for predicting damaging missense mutations. Nat Methods 2010, 7:248-9.), SIFT (http://sift.jcvi.org and referenced in Kumar P et al., “Predicting the effects of coding non-synonymous variants on protein function using the SIFT algorithm”, Nat Protoc.—2009; Vol. No.4(7), pp.:1073-81) and PROVEAN (http://provean.jcvi.org/index.php and referenced in Choi Y et al., “Predicting the Functional Effect of Amino Acid Substitutions and Indels”, PLoS ONE—2012, Vol. No. 7(10): e46688.). When the mean frequency of an alternative allele on the dog population analysed was more than 0.25, both alleles of those SNPs were tested with algorithms mentioned before.

SMART program was used in order to identify protein domains of each TLR (Letunic I, Doerks T, Bork P: SMART 7: recent updates to the protein domain annotation resource. Nucleic Acids Res 2012, 40(Database issue):D302-5). Multialignments to nearest species was performed with Multialin platform (http://multalin.toulouse.inra.fr/multalin/).

With this approximation, materials and methods, there were detected known mutations as well as other new mutations, all of them identified in Table 1 of Example 2.

### Example 2

### DNA Array Comprising Canine tlr Gene Fragments Capable of Detecting Critical Mutations in Canine TLR Proteins

An array of type TaqMan OpenArray® was designed. Selected SNPs and their surrounding sequences, 60 nucleotides upstream and 60 nucleotides downstream were introduced in Custom TaqMan® Assay Design Tool web site (www.appliedbiosystems.com/cadt) from Life Technologies® to validate if the sequences were suitable for TaqMan assay design. Other SNPs in the context sequences were indicated with an “N” before the assay designs.

The array comprised oligonucleotides with a length from 10 to 30 nucleotides, which were tlr gene fragments and capable of identifying the mutations indicated in next Table 1.

TABLE 1
Non-synonymous SNPs and frameshift mutations of canine TLRs in
the TaqMan Open Array plate designed.

| | | Chr:bp | | | Previous | |
|---|---|---|---|---|---|---|
| Canine gene | SNP | position | dbSNP ID | AA Subst | detected^{a} | Validated?^{b} |
| TLR1 | G/T | 3:73542337 | rs23585044 | S29I | Massive seq | YES |
| | T/G | 3:73543092 | new | S281A | Massive seq | YES |
| | G/A | 3:73543185 | new | V312I | Massive seq | YES |
| | T/A | 3:73544153 | rs23572381 | N634K¹ | CanFam 3.1 | YES² |
| | T/A | 3:73544153 | rs23572381 | N634K¹ | CanFam 3.1 | NO |
| | G/A | 3:73544221 | rs23572380 | S657N | CanFam 3.1 | YES² |
| TLR2 | C/A | 15:51463020 | rs22410121 | S46Y | Massive seq | YES |
| | A/0 | 15:51464076 | rs8958543 | A398- | CanFam 3.1 | YES² |
| | C/T | 15:51464430 | new | S516L | Massive seq | YES |
| | C/T | 15:51464700 | new | T606M | Massive seq | YES |
| TLR3 | C/G | 16:.44623632 | new | E176D | Massive seq | YES |
| TLR4 | T/C | 11:71356420 | rs22120766 | V8A | Massive seq | NO |
| | G/C | 11:71360743 | rs22157966 | A34P | Massive seq | YES |
| | G/A | 11:71360887 | new | V82M | Massive seq | YES |
| | T/C | 11:71364581 | rs22145736 | L167P | Massive seq | YES |
| | C/A | 11:71364681 | rs22189454 | H200Q | Massive seq | YES |
| | A/G | 11:71364769 | rs22189456 | K230E | Massive seq | YES |
| | G/A | 11:71365120 | new | A347T | Massive seq | YES |
| | A/T | 11:71365652 | rs22124023 | E524V | Massive seq | YES |
| | A/G | 11:71365810 | new | T577A | Massive seq | YES |
| | G/A | 11:71365888 | rs22123995 | E603K | Massive seq | YES |
| TLR5 | G/A | 38:23702193 | rs24029590 | G54E | CanFam 3.1 | NO |
| | 0/C | 38:23702251 | rs9070448 | -74C | CanFam 3.1 | YES² |
| | A/G | 38:23702514 | rs9070450 | Y161C | CanFam 3.1 | YES |
| | A/C | 38:23702539 | new | E169D | Massive seq | YES |
| | G/A | 38:23702562 | new | S177N | Massive seq | YES |
| | G/C | 38:23702640 | rs9070451 | R203P | Massive seq | YES |
| | T/C | 38:23702684 | rs9070452 | W218R | Massive seq | NO |
| | C/T | 38:23702837 | rs9070447 | R269C | Massive seq | YES |
| | G/A | 38:23702918 | new | V296I | Massive seq | YES |
| | T/C | 38:23703180 | new | L383S | Massive seq | YES |
| | G/A | 38:23703237 | new | R402Q | Massive seq | YES |
| | G/A | 38:23703279 | new | R416Q | Massive seq | YES |
| | T/0 | 38:23703591 | rs9125247 | T520- | CanFam 3.1 | YES² |
| | G/A | 38:23703629 | new | G533S | Massive seq | YES |
| | G/A | 38:23704233 | new | R734Q | Massive seq | YES |
| | G/T | 38:23704331 | new | D767Y | Massive seq | YES |
| | C/T | 38:23704562 | new | R844C | Massive seq | YES |
| | T/C | 38:23704581 | rs24029975 | L850S | Massive seq | YES |
| | T/G | 38:23704695 | new | F888C | Massive seq | YES |
| | G/A | 38:23704718 | new | A896T | Massive seq | YES |
| | C/T | 38:23705081 | new | H1017Y | Massive seq | YES |
| | G/A | 38:23705090 | new | G1020S | Massive seq | YES |
| | G/A | 38:23705178 | new | R1049Q | Massive seq | YES |
| | G/A | 38:23705264 | new | A1078T | Massive seq | YES |
| TLR6 | A/G | 3:73521250 | new | Y182C | Massive seq | YES |
| | C/T | 3:73522074 | new | L457F | Massive seq | YES |
| | G/A | 3:73522242 | rs23570247 | D513N | Massive seq | YES |
| TLR7 | C/G | X:9334108 | new | A16G | Massive seq | YES² |
| | C/A | X:9355727 | new | F167L | Massive seq | YES |
| | C/T | X:9358423 | new | P1066L | Massive seq | YES |
| TLR8 | T/C | X:9397240 | new | V157A | Massive seq | YES |
| | G/A | X:9397663 | new | R298Q | Massive seq | YES |
| | G/A | X:9398094 | rs24607342 | G442S | Massive seq | YES |
| | G/A | X:9398827 | rs24607358 | R686H | Massive seq | YES |
| TLR9 | G/A | 20:37544129 | new | V87I | Massive seq | YES |
| | 0/A | 20:37544851 | rs9188882 | -328A | CanFam 3.1 | YES² |
| | A/G | 20:37545011 | new | K381E | Massive seq | YES |
| | C/A | 20:37545245 | new | P459T | Massive seq | YES |
| | A/G | 20:37546031 | rs22882109 | S721G | Massive seq | YES |
| | C/T | 20:37546230 | new | P787L | Massive seq | ND³ |
| | C/T | 20:37546454 | new | R862W | Massive seq | YES |
| TLR10 | C/T | 3:73569402 | rs23518574 | T361M | Massive seq | YES |
| | A/G | 3:73570094 | new | M592V | Massive seq | YES |

^{a}Previous detected by massive sequencing, which can also imply data retrieved from CanFam 3.1 database, only when dbSNP ID is specified. CanFam 3.1 means that SNP was not seen by massive sequencing, but in databases. Always concerning this study.
^{b}Validated assays (YES) or not (NO) by TagMan Open Array.
¹SNP considered twice with a different surrender SNP in order to detect it.
²Assay has been validated technically, although not genetically because all individuals present only the reference allele.
³ND (not determined),
Chr:bp position means Chromosome of canis lupus familiaris: base pair (bp) position.
dbSNP ID means database single nucleotide polymorphism identification, from NCBI SNps database retrievable from http://www.ncbi.nlm.nih.gov/snp/
AA Subst means amino acid substitution in the polypeptide sequence of TLR.

Analysis of PCR results was performed with the TaqMan Genotyper software v.1.3 (Applied Biosystems).

Probes for detecting new non-synonymous mutations included in the array were those indicated in Table 2:

TABLE 2
Probes in a TaqMan type array for
detecting new non-synonymous mutations
in canine TLR genes and proteins.

| Canine | | |
|---|---|---|
| gene/ | | |
| Canine | AA | |
| protein | subst | Probe sequence |
| tlr 1/TLR1 | S281A | CATTTGAAATTGAGAAATAC |
| | | (SEQ ID NO: 14) |
| tlr 2/TLR2 | S516L | CTCCTTCGAGAAAGC |
| | | (SEQ ID NO: 11) |
| tlr 2/TLR2 | T606M | CCCGCCGTGAGCAG |
| | | (SEQ ID NO: 16) |
| tlr 3/TLR3 | E176D | TCTCCAAGAGCTTCTG |
| | | (SEQ ID NO: 17) |
| tlr 4/TLR4 | V82M | CAAAACTGCAGGTGCTGG |
| | | (SEQ ID NO: 18) |
| tlr 4/TLR4 | A347T | CCCATGCGGGAAAT |
| | | (SEQ ID NO: 19) |
| tlr 4/TLR4 | T577A | AAGTTATTCCGAGTAAGATT |
| | | (SEQ ID NO: 20) |
| tlr 5/TLR5 | F888C | CCAGGAGGAACAGCGT |
| | | (SEQ ID NO: 47) |
| tlr 5/TLR5 | E169D | TTCCCCGAAGCCCTG |
| | | (SEQ ID NO: 21) |
| tlr 5/TLR5 | S177N: | TGTTTGCCGTCACTCAA |
| | | (SEQ ID NO: 22) |
| tlr 5/TLR5 | V296I | TCCGGGCCGTCACC |
| | | (SEQ ID NO: 23) |
| tlr 5/TLR5 | L383S | AGGCGCAACAAAG |
| | | (SEQ ID NO: 24) |
| tlr 5/TLR5 | R402Q | TCCTTCCGGGAGCTG |
| | | (SEQ ID NO: 25) |
| tlr 5/TLR5 | R416Q | CTCAACCGGATCCC |
| | | (SEQ ID NO: 26) |
| tlr 5/TLR5 | G533S | TCGCACGGCTTCG |
| | | (SEQ ID NO: 27) |
| tlr 5/TLR5 | R734Q | AGCCCCCGGAACAC |
| | | (SEQ ID NO: 28) |
| tlr 5/TLR5 | D767Y | TCAGGTCGAGGCCC |
| | | (SEQ ID NO: 29) |
| tlr 5/TLR5 | R844C | CGCAGCGCACGTC |
| | | (SEQ ID NO: 30) |
| tlr 5/TLR5 | A896T | CTCGTGGCCGCCAAG |
| | | (SEQ ID NO: 31) |
| tlr 5/TLR5 | H1017Y | CCAGGTGGGACGCG |
| | | (SEQ ID NO: 32) |
| tlr 5/TLR5 | G1020S | CCTGGACGGCGCCCT |
| | | (SEQ ID NO: 33) |
| tlr 5/TLR5 | R1049Q | CAGCGCCGGTACTT |
| | | (SEQ ID NO: 34) |
| tlr 5/TLR5 | A1078T | CGCGCGCCCCTC |
| | | (SEQ ID NO: 35) |
| tlr 6/TLR6 | L457F | TTATCGTGAAGATCAAGC |
| | | (SEQ ID NO: 12) |
| tlr 6/TLR6 | Y1820 | TTCTTTTGCATAATAACC |
| | | (SEQ ID NO: 36) |
| tlr 7/TLR7 | A16G | AGGGAGCCTGCTTCT |
| | | (SEQ ID NO: 37) |
| tlr 7/TLR7 | F167L | TGCATCGGAAATCG |
| | | (SEQ ID NO: 38) |
| tlr 7/TLR7 | P1066L | CTTGAGAAGCCCCTTCAG |
| | | (SEQ ID NO: 39) |
| tlr 8/TLR8 | V157A | CATCATCTGGGTAACGAA |
| | | (SEQ ID NO: 13) |
| tlr 8/TLR8 | R298Q | CCCTCCGGAAGATT |
| | | (SEQ ID NO: 40) |
| tlr 9/TLR9 | V87I | TTGTCCACTTCGTCCACC |
| | | (SEQ ID NO: 41) |
| tlr 9/TLR9 | K381E | CGCTCAGCAAGACCA |
| | | (SEQ ID NO: 42) |
| tlr 9/TLR9 | P459T | CCCAGTGGGCCCAGAG |
| | | (SEQ ID NO: 43) |
| tlr 9/TLR9 | P787L | ACGGCTAGGCAGGCC |
| | | (SEQ ID NO: 44) |
| tlr 9/TLR9 | R862W | CCCGCCGCCGCC |
| | | (SEQ ID NO: 45) |
| tlr 10/TLR10 | M592V | CCACAGCCATCCCCAG |
| | | (SEQ ID NO: 46) |

In addition, the array included the pair of primers for amplifying any tlr gene fragment to which the probes for identifying the above cited new mutations could hybridize. In particular, it included primers of SEQ ID NO: 53 to SEQ ID NO: 128.

The array also comprised specially designed TaqMan probes for the rest of mutations identified in Table 1 (i.e. frameshifts mutations and other known non-synonymous mutations). In addition, in order to detect also wild-type alleles of the tlr genes, the array comprised TaqMan probes designed for detecting wild-type canine tlr gene fragments. These probes were used as positive controls and allowed the detection of heterozygote individuals for said feature or character (i.e. a particular mutation in a TLR protein), as well as the detection of homozygote individuals.

Canine tlr gene fragments in the array that were acting as probes incorporated a 5′ reporter dye (VIC for probes for detecting mutations, and FAM for probes viewing wild-type or non-mutated canine TLR from a sample), and a 3′ nonfluorescent quencher.

In summary, the array according to the invention (from the type TaqMan OpenArray® plate) was developed for the detection and for the validation of the nsSNPs (non-synonymous mutations) by individual genotyping (Table 1). The panel contained (i) 27 out of 31 nsSNPs that were predicted to have an impact on the protein structure (4 wolf-specific SNPs were not considered for the array: TLR1 A525V, TLR5 N833K, TLR6 P579L and TLR10 F787L; see Table 3); (ii) 28 out of the 33 remaining nsSNPs segregating in dogs (5 SNPs that were not suitable for a correct primer design were rejected for posterior analysis: TLR4 T36A, TLR4 T36I, TLR5 T243A, TLR5 Q213R and TLR9 A442V); and (iii) 4 frameshift and 4 non-synonymous TLR polymorphisms described on CanFam 3.1 but not detected in the analysed cohorts. One of the non-synonymous variants added (rs23572381, TLR1 N634K) was designed with two different TaqMan assays due to the presence of other variants close to the interrogated SNP.

Table 1 allows concluding that all newly identified mutations can be detected simultaneously in a DNA array specially designed for the study of relevant mutations in canine tlr genes and the corresponding TLR expressed proteins. Detection of these mutations may be useful in defining the innate immunity profile; for determining the genetic predisposition of an individual of Canis genera to suffer from any disease related with dysfunctions of the innate immunity, for determining variability among races and among individuals into a specific race for example with the aim of homogenising animal cohorts for experimental purposes; and for the unequivocally individual identification of samples.

This particular DNA array represents an interesting tool for genotyping an individual subject of the Canis genera (especially dogs), with the aim of finally determining if this individual is susceptible of suffering from any disease related with TLR dysfunctions.

Association of the detected mutations with the susceptibility of suffering from any disease related with TLR dysfunctions is derivable from next Table 3, wherein the impact of newly identified and known canine TLRs Amino Acid substitutions was predicted using bioinformatics tools.

TABLE 3
Summary data for non-synonymous SNPs predicted to impact protein function either by Polyphen-2, SIFT or PROVEAN

| | | | | | | | | | Variant | Variant |
|---|---|---|---|---|---|---|---|---|---|---|
| | | | | AA | Protein | Polyphen-2 | | Provean | freq | freq |
| Canine gene | Position | SNP | dbSNP ID | Subst^{a} | domain^{b} | result^{c} | SIFT result^{c} | result^{c} | (dog)^{d} | (wolf)^{e} |
| EXTRACELLULAR TLRs | | | | | | | | | | |
| TLR1 | 3:73542337 | G/T | rs23585044 | S29I | ncp | Pos. damaging | Tolerated | Neutral | 0.36 | 0.77 |
| | 3:73543092 | T/G | new | S281A | ncp | Pos. damaging | Tolerated | Neutral | 0.06 | 0.00 |
| | 3:73543825 | C/T | new | A525V | LRRCT² | Pos. damaging | Tolerated | Deleterious | 0 | 0.11 |
| TLR2 | 15:51463020 | C/A | rs22410121 | S46Y | ncp | Pos. damaging | Tolerated | Neutral | 0.10 | 0.00 |
| | 15:51464430 | C/T | new | S516L | ncp | Prob. damaging | Aff. function | Deleterious | 0.14 | 0 |
| TLR3 | 16:44623632 | C/G | new | E176D | ncp | Pos. damaging | Tolerated | Neutral | 0.16 | 0.12 |
| TLR4 | 11:71356420 | C/T | reference¹ | A8V | ncp | Prob. damaging | Tolerated | Neutral | 0.77 | 0.57 |
| | 11:71360887 | G/A | new | V82M | ncp | Pos. damaging | Tolerated | Neutral | 0.09 | 0.15 |
| | 11:71364581 | T/C | rs22145736 | L167P | ncp | Prob. damaging | Aff. function | Deleterious | 0.15 | 0 |
| | 11:71364681 | A/C | reference¹ | Q200H | ncp | Pos. damaging | Aff. function | Neutral | 0.88 | 0.23 |
| | 11:71365810 | A/G | new | T577A | LRRCT³ | Pos. damaging | Aff. function | Neutral | 0.01 | 0 |
| TLR5 | 38:23702837 | C/T | rs9070447 | R269C | ncp | Prob. damaging | Aff. function | Neutral | 0.19 | 0.01 |
| | 38:23702918 | G/A | new | V296I | ncp | Pos. damaging | Tolerated | Neutral | 0.05 | 0.26 |
| | 38:23703629 | G/A | new | G533S | ncp | Prob. damaging | Tolerated | Neutral | 0.02 | 0 |
| | 38:23704331 | G/T | new | D767Y | ncp | Prob. damaging | Tolerated | Deleterious | 0.04 | 0 |
| | 38:23704531 | C/G | new | N833K | LRRCT | Pos. damaging | Tolerated | Deleterious | 0 | 0.06 |
| | 38:23704562 | C/T | new | R844C | LRRCT | Pos. damaging | Tolerated | Neutral | 0.04 | 0.39 |
| | 38:23704581 | C/T | reference¹ | S850L | LRRCT | Prob. damaging | Aff. function | Deleterious | 0.68 | 0.02 |
| | 38:23704695 | T/G | new | F888C | low | Prob. damaging | Aff. function | Deleterious | 0.04 | 0 |
| | | | | | complexity | | | | | |
| | 38:23705081 | C/T | new | H1017Y | TIR | Benign | Aff. function | Neutral | 0.02 | 0 |
| | 38:23705264 | G/A | new | A1078T | TIR⁴ | Pos. damaging | Aff. function | Neutral | 0.07 | 0.00 |
| TLR6 | 3:73521250 | A/G | new | Y182C | ncp | Prob. damaging | Tolerated | Deleterious | 0.01 | 0.09 |
| | 3:73522074 | C/T | new | L457F | ncp | Prob. damaging | Aff. function | Deleterious | 0.03 | 0 |
| | 3:73522242 | G/A | rs23570247 | D513N | ncp | Pos. damaging | Tolerated | Neutral | 0.73 | 1.00 |
| | 3:73522441 | C/T | new | P579L | LRRCT | Pos. damaging | Aff. function | Deleterious | 0.01 | 0.07 |
| TLR10 | 3:73569402 | C/T | rs23518574 | T361M | ncp | Prob. damaging | Aff. function | Deleterious | 0.13 | 0.12 |
| | 3:73570681 | T/A | new | F787L | TIR⁵ | Pos. damaging | Low | Neutral | 0.00 | 0.39 |
| | | | | | | | confidence | | | |
| INTRACELLULAR TLRs | | | | | | | | | | |
| TLR8 | X:9397240 | T/C | new | V157A | ncp | Pos. damaging | Aff. function | Deleterious | 0.06 | 0 |
| TLR9 | 20:37544129 | G/A | new | V87I | ncp | Benign | Aff. function | Neutral | 0.02 | 0 |
| | 20:37546230 | C/T | new | P787L | ncp | Pos. damaging | Tolerated | Neutral | 0.22 | 0.24 |
| | 20:37546454 | C/T | new | R862W | ncp | Prob. damaging | Tolerated | Neutral | 0.2 | 0 |

ENSEMBL protein ID: ENSCAFP00000032660 (TLR1); ENSCAFP00000012269 (TLR2); ENSCAFP00000011004 (TLR3); ENSCAFP00000031395 and ENSCAFP00000031396 (TLR4); ENSCAFP00000016726 (TLR5); ENSCAFP00000023836 (TLR6); ENSCAFP00000031505 (TLR8); ENSCAFP00000030804 (TLR9); ENSCAFP00000023840 (TLR10)
^{a}Amino acid (AA) substitutions predicted from reference proteins (ENSEMBL Protein ID)
^{b}Protein domain locations predicted by SMART (http://smart.embl-heidelberg.de/). Only confidently predicted domains are depicted [ncp, no confident prediction]
^{c}Results from PolyPhen-2, SIFT and PROVEAN(41, 42, X). Results from Polyphen-2 can be benign, possibly damaging or probably damaging. From SIFT they can either be tolerated or affect protein function.
^{d}Observed frequency of nonsynonymous SNP allele in all 350 dogs from 7 different breeds by massive sequencing.
^{e}Observed frequency of nonsynonymous SNP allele in 100 wolves by massive sequencing.
¹reference allele tested as the alternative in the SNP
²Leucine Rich Repeat C-terminal (LRRCT) domain predicted from aminoacid 528 to 582.
³LRRCT domain predicted from aminoacid 579 to 629.
⁴TIR domain predicted from aminoacid 927 to 1074.
⁵TIR domain predicted from aminoacid 641 to 784.

Polyphen-2, SIFT and PROVEAN tools were used in order to predict the effect of each new non-synonymous SNP (nsSNPs) in the protein structure (Table 3). 33 out of 64 nsSNPs were predicted as benign, tolerated and neutral using the three algorithms mentioned. 31 out of 64 nsSNPs were predicted to have an effect on protein structure by at least one of the tools used.

Results from Polyphen-2, SIFT and PROVEAN were convergent in predicting damaging effects of 8 out of 31 nsSNPs (27%). 6 out of 64 were not correctly predicted, giving unknown or low confidence results, because they were not aligning to enough similar sequences to give a reliable result. Curiously those 6 nsSNPs were all located on N-terminal of TLR5. Suggesting that those first aminoacids were not part of TLR5 of other species, perhaps due to a bad annotation of this protein (discussion)

Frequencies of nsSNPs on table 3 are an average of all dog breeds tested and both wolf populations respectively, so all variants are polymorphic at least in one breed (minimum allele frequency MAF>0.05). Looking at frequencies of nsSNPs predicted to be damaging in canine TLRs, it can be seen that 17 out of 31 show a MAF>0.05 (15 out of 31 with MAF>0.05 in wolf populations).

Protein structure of TLRs was assessed using SMART (Letunic I, Doerks T, Bork P (2012) SMART 7: recent updates to the protein domain annotation resource. Nucleic Acids Res 40:D302-305), which predicts domains taking into account aminoacid sequences. 6 out of 31 nsSNPs predicted to be damaging in canine TLRs were found to be in a Leucine Rich Repeat C-terminal (LRRCT) or really close to it. Only 1 out of 31 was found to affect TIR domain in TLR 5, another 2 were found to be really close to this domain in TLR5 and 10. With the exception of these last ones, damaging SNPs were in most cases located in the sensor domain of TLRs.

### Example 3

### Individual Identification of Dogs from a Beagle Cohort and Other Breeds using the Array of the Invention

Finally, in order to show the application of the array of the invention for unequivocally identifying an individual among a group (even of the same breed), the inventors compared the data derived from DNA samples of the 15 Beagles. The array comprised, as above indicated, the probes of Table 2, these probes are indeed tlr gene fragments, which in the corresponding tlr gene, are codifying for some mutations. The array also comprised other probes and means for the detection of the other mutations listed in Table 1.

The read of the nucleotides in all these possibly mutated points of TLR genes and corresponding proteins using the array of the invention, revealed enough information from each individual for distinguishing between two animals.

Results were also confirmed in other breeds such as German Shepherd, Labrador, Boxer, Yorkshire terrier, French bulldog and Shar-pei.

Data from Table 4 correspond to the nucleotides detected in both alleles of each gene at the positions corresponding to possible mutated points in tlr genes.

TABLE 4
Individual TLR signature of a Beagle cohort (table divided in 15 parts
from 4.1 to 4.15, each containing 4 mutation points)
Table 4.1

| Breed | Sample ID | TLR1_S29I_chr3_7 | TLR1_S281A_chr3_ | TLR1_V312I_chr3_ | TLR1_N634K_rs235 |
|---|---|---|---|---|---|
| Beagle | BGL_8218 | G T | T T | G G | T T |
| Beagle | BGL_8220-conc | G T | T T | G G | T T |
| Beagle | BGL_8222 | T T | T T | G G | T T |
| Beagle | BGL_8224 | G T | T T | G G | T T |
| Beagle | BGL_8226 | T T | T T | G G | T T |
| Beagle | BGL_8272 | G T | T T | G G | T T |
| Beagle | BGL_8276 | G T | T T | G G | T T |
| Beagle | BGL_8278 | T T | T T | G G | T T |
| Beagle | BGL_8280-50 | T T | T T | G G | T T |
| Beagle | BGL_8284 | T T | T T | G G | T T |
| Beagle | BGL_8286 | T T | T T | G G | T T |
| Beagle | BGL_8288 | T T | T T | G G | T T |
| Beagle | BGL_8290-50 | T T | T T | G G | T T |
| Beagle | BGL_8292 | T T | T T | G G | T T |
| Beagle | BGL_8294 | T T | T T | G G | T T |

Table 4.2

| Breed | Sample ID | TLR1_S657N_rs235 | TLR2_S46Y_chr15_ | TLR2_S5161_chr15 | TLR2_T606M_chr15 |
|---|---|---|---|---|---|
| Beagle | BGL_8218 | G G | C C | C C | C C |
| Beagle | BGL_8220-conc | G G | C C | C C | C C |
| Beagle | BGL_8222 | G G | C C | C C | C C |
| Beagle | BGL_8224 | G G | C C | C C | C C |
| Beagle | BGL_8226 | G G | C C | C C | C C |
| Beagle | BGL_8272 | G G | C C | C C | C C |
| Beagle | BGL_8276 | G G | C C | C C | C C |
| Beagle | BGL_8278 | G G | C C | C C | C C |
| Beagle | BGL_8280-50 | G G | C C | C C | C C |
| Beagle | BGL_8284 | G G | C C | C C | C C |
| Beagle | BGL_8286 | G G | C C | C C | C C |
| Beagle | BGL_8288 | G G | C C | C C | C C |
| Beagle | BGL_8290-50 | G G | C C | C C | C C |
| Beagle | BGL_8292 | G G | C C | C C | C C |
| Beagle | BGL_8294 | G G | C C | C C | C C |

Table 4.3

| Breed | Sample ID | TLR2_398_FRAMESH | TLR3_E176D_chr16 | TLR4_A34P_chr11_ | TLR4_V82M_chr11_ |
|---|---|---|---|---|---|
| Beagle | BGL_8218 | A A | C C | G G | G G |
| Beagle | BGL_8220-conc | A A | C C | G G | G G |
| Beagle | BGL_8222 | A A | C C | G G | G A |
| Beagle | BGL_8224 | A A | C C | G G | G G |
| Beagle | BGL_8226 | A A | C C | G G | G A |
| Beagle | BGL_8272 | A A | C C | G G | G A |
| Beagle | BGL_8276 | A A | C G | G G | G G |
| Beagle | BGL_8278 | A A | C C | G G | G G |
| Beagle | BGL_8280-50 | A A | C C | G G | G G |
| Beagle | BGL_8284 | A A | C C | G G | G G |
| Beagle | BGL_8286 | A A | C C | G G | G G |
| Beagle | BGL_8288 | A A | C C | G G | G G |
| Beagle | BGL_8290-50 | A A | C C | G G | G G |
| Beagle | BGL_8292 | A A | C C | G G | G G |
| Beagle | BGL_8294 | A A | C G | G G | G G |

Table 4.4

| Breed | Sample ID | TLR4_L167P_chr11 | TLR4_H200Q_chr11 | TLR4_K230E_chr11 | TLR4_A347T_chr11 |
|---|---|---|---|---|---|
| Beagle | BGL_8218 | T T | C C | A A | A A |
| Beagle | BGL_8220-conc | T T | C C | A A | A A |
| Beagle | BGL_8222 | T T | C C | A A | A A |
| Beagle | BGL_8224 | T T | C C | A A | A A |
| Beagle | BGL_8226 | T T | C C | A A | A A |
| Beagle | BGL_8272 | T T | C C | A A | A A |
| Beagle | BGL_8276 | T T | C C | A A | G A |
| Beagle | BGL_8278 | T T | C C | A A | A A |
| Beagle | BGL_8280-50 | T T | C C | A A | A A |
| Beagle | BGL_8284 | T T | C C | A A | A A |
| Beagle | BGL_8286 | T T | C C | 0 0 | A A |
| Beagle | BGL_8288 | T T | C C | A A | A A |
| Beagle | BGL_8290-50 | T T | C C | A A | A A |
| Beagle | BGL_8292 | T C | C C | A A | G A |
| Beagle | BGL_8294 | T T | C C | A A | A A |

Table 4.5

| Beagle | Sample ID | TLR4_E524V_chr11 | TLR4_T577A_chr11 | TLR4_E603K_chr11 | TLR5_E169D_chr38 |
|---|---|---|---|---|---|
| Beagle | BGL_8218 | T T | A A | A A | A C |
| Beagle | BGL_8220-conc | T T | A A | A A | 0 0 |
| Beagle | BGL_8222 | T T | A A | A A | A C |
| Beagle | BGL_8224 | T T | A A | A A | A C |
| Beagle | BGL_8226 | T T | A A | A A | A A |
| Beagle | BGL_8272 | T T | A A | A A | C C |
| Beagle | BGL_8276 | A T | A A | G A | A C |
| Beagle | BGL_8278 | T T | A A | A A | A C |
| Beagle | BGL_8280-50 | T T | A A | A A | A C |
| Beagle | BGL_8284 | T T | A A | A A | A C |
| Beagle | BGL_8286 | T T | A A | A A | C C |
| Beagle | BGL_8288 | T T | A A | A A | C C |
| Beagle | BGL_8290-50 | T T | A A | A A | A C |
| Beagle | BGL_8292 | A T | A A | G A | A C |
| Beagle | BGL_8294 | T T | A A | A A | A C |

Table 4.6

| Breed | Sample ID | TLR5_S177N_chr38 | TLR5_R203P_chr38 | TLR5_R269C_chr38 | TLR5_V296I_chr38 |
|---|---|---|---|---|---|
| Beagle | BGL_8218 | G A | G G | C C | G G |
| Beagle | BGL_8220-conc | G A | G C | C C | G G |
| Beagle | BGL_8222 | G A | G G | C C | G G |
| Beagle | BGL_8224 | G G | C C | C C | G G |
| Beagle | BGL_8226 | G G | G C | C C | G G |
| Beagle | BGL_8272 | G A | G C | C C | G G |
| Beagle | BGL_8276 | G A | G C | C C | G G |
| Beagle | BGL_8278 | G G | G C | C C | G G |
| Beagle | BGL_8280-50 | G G | G C | C C | G G |
| Beagle | BGL_8284 | G G | C C | C C | G G |
| Beagle | BGL_8286 | G G | C C | C C | G G |
| Beagle | BGL_8288 | G G | C C | C C | G G |
| Beagle | BGL_8290-50 | G G | G C | C C | G G |
| Beagle | BGL_8292 | G G | G C | C C | G G |
| Beagle | BGL_8294 | G G | C C | C C | G G |

Table 4.7

| Breed | Sample ID | TLR5_L383S_chr38 | TLR5_R402Q_chr38 | TLR5_R416Q_chr38 | TLR5_G533S_chr38 |
|---|---|---|---|---|---|
| Beagle | BGL_8218 | T C | G G | G G | G G |
| Beagle | BGL_8220-conc | C C | G G | G A | G G |
| Beagle | BGL_8222 | T C | G G | G G | G G |
| Beagle | BGL_8224 | C C | G G | G A | G G |
| Beagle | BGL_8226 | T C | G G | G G | G G |
| Beagle | BGL_8272 | C C | G G | G A | G G |
| Beagle | BGL_8276 | C C | G G | G G | G G |
| Beagle | BGL_8278 | C C | G G | G G | G G |
| Beagle | BGL_8280-50 | T C | G G | G G | G G |
| Beagle | BGL_8284 | C C | G G | G G | G G |
| Beagle | BGL_8286 | C C | G G | G G | G G |
| Beagle | BGL_8288 | C C | G G | G G | G G |
| Beagle | BGL_8290-50 | T C | G G | G G | G G |
| Beagle | BGL_8292 | T C | G G | G G | G G |
| Beagle | BGL_8294 | C C | G G | G G | G G |

Table 4.8

| Breed | Sample ID | TLR5_R734Q_chr38 | TLR5_D767Y_chr38 | TLR5_R844C_chr38 | TLR5_L850S_chr38 |
|---|---|---|---|---|---|
| Beagle | BGL_8218 | G G | G G | C C | T T |
| Beagle | BGL_8220-conc | G G | G G | C C | T C |
| Beagle | BGL_8222 | G G | G G | C C | T T |
| Beagle | BGL_8224 | G G | G G | C C | C C |
| Beagle | BGL_8226 | G G | G G | C C | T C |
| Beagle | BGL_8272 | G G | 3 G | C C | T T |
| Beagle | BGL_8276 | G G | G G | C C | C C |
| Beagle | BGL_8278 | G G | G G | C C | C C |
| Beagle | BGL_8280-50 | G G | G G | C C | T C |
| Beagle | BGL_8284 | G G | G G | C C | C C |
| Beagle | BGL_8286 | G G | G G | C C | C C |
| Beagle | BGL_8288 | G G | G G | C C | C C |
| Beagle | BGL_8290-50 | G G | G G | C C | T C |
| Beagle | BGL_8292 | G G | G G | C C | T C |
| Beagle | BGL_8294 | G G | G G | C C | C C |

Table 4.9

| Breed | Sample ID | TLR5_F888C_chr38 | TLR5_A896T_chr38 | TLR5_H1017Y_chr3 | TLR5_G1020S_chr3 |
|---|---|---|---|---|---|
| Beagle | BGL_8218 | T T | G G | C C | G G |
| Beagle | BGL_8220-conc | T T | G A | C T | G G |
| Beagle | BGL_8222 | T T | G G | C C | G G |
| Beagle | BGL_8224 | T T | G A | C T | G G |
| Beagle | BGL_8226 | T T | G G | C C | G G |
| Beagle | BGL_8272 | T T | G A | C T | G G |
| Beagle | BGL_8276 | T T | G G | C C | G G |
| Beagle | BGL_8278 | T T | G G | C C | G G |
| Beagle | BGL_8280-50 | T T | G G | C C | G A |
| Beagle | BGL_8284 | T T | G G | C C | G G |
| Beagle | BGL_8286 | T T | G G | C C | G A |
| Beagle | BGL_8288 | T T | G G | C C | G G |
| Beagle | BGL_8290-50 | T T | G G | C C | G G |
| Beagle | BGL_8292 | T T | G G | C C | G G |
| Beagle | BGL_8294 | T T | G G | C C | G A |

Table 4.10

| Breed | Sample ID | TLR5_R1049Q_chr3 | TLR5_A1078T_chr3 | TLR5_Y161C_rs907 | TLR5_73_74_FRAME |
|---|---|---|---|---|---|
| Beagle | BGL_8218 | G G | G G | A A | * * |
| Beagle | BGL_8220-conc | G G | G A | A A | * * |
| Beagle | BGL_8222 | G G | G G | A A | * * |
| Beagle | BGL_8224 | G G | G A | A A | * * |
| Beagle | BGL_8226 | G G | G G | A A | * * |
| Beagle | BGL_8272 | G G | G A | A A | * * |
| Beagle | BGL_8276 | G G | G G | A A | * * |
| Beagle | BGL_8278 | G G | G G | A A | * * |
| Beagle | BGL_8280-50 | G A | G G | A A | * * |
| Beagle | BGL_8284 | G G | G G | A A | * * |
| Beagle | BGL_8286 | G A | G G | G G | * * |
| Beagle | BGL_8288 | G G | G G | G G | * * |
| Beagle | BGL_8290-50 | G G | G G | A A | * * |
| Beagle | BGL_8292 | G G | G G | A A | * * |
| Beagle | BGL_8294 | G A | G G | A A | * * |

Table 4.11

| Breed | Sample ID | TLR5_520_FRAMESH | TLR6_Y182C_chr3_ | TLR6_L457F_chr3_ | TLR6_D513N_chr3_ |
|---|---|---|---|---|---|
| Beagle | BGL_8218 | T T | A A | C C | G G |
| Beagle | BGL_8220-conc | T T | A A | C C | G G |
| Beagle | BGL_8222 | 0 0 | A A | C C | G G |
| Beagle | BGL_8224 | T T | A A | C C | G G |
| Beagle | BGL_8226 | T T | A A | C C | G G |
| Beagle | BGL_8272 | T T | A A | C C | G G |
| Beagle | BGL_8276 | T T | A A | C C | G G |
| Beagle | BGL_8278 | T T | A A | C C | G G |
| Beagle | BGL_8280-50 | T T | A A | C C | A A |
| Beagle | BGL_8284 | T T | A A | C C | G A |
| Beagle | BGL_8286 | T T | A A | C C | G G |
| Beagle | BGL_8288 | 0 0 | A A | C C | G A |
| Beagle | BGL_8290-50 | T T | A A | C C | G G |
| Beagle | BGL_8292 | T T | A A | C C | G G |
| Beagle | BGL_8294 | T T | A A | C C | G G |

Table 4.12

| Breed | Sample ID | TLR7_A16G_chrX_9 | TLR7_F1671_chrX_ | TLR7_P1066L_chrX | TLR8_V157A_chrX_ |
|---|---|---|---|---|---|
| Beagle | BGL_8218 | C C | C C | C C | T T |
| Beagle | BGL_8220-conc | C C | C C | C C | T T |
| Beagle | BGL_8222 | C C | C C | C C | T T |
| Beagle | BGL_8224 | C C | C C | C C | T T |
| Beagle | BGL_8226 | C C | C C | C C | T T |
| Beagle | BGL_8272 | C C | C C | C C | T T |
| Beagle | BGL_8276 | C C | C C | C C | T T |
| Beagle | BGL_8278 | C C | C C | C C | T T |
| Beagle | BGL_8280-50 | C C | C C | C C | T T |
| Beagle | BGL_8284 | C C | C C | C C | T T |
| Beagle | BGL_8286 | C C | C C | C C | T T |
| Beagle | BGL_8288 | C C | C C | C C | T T |
| Beagle | BGL_3290-50 | C C | C C | C C | T T |
| Beagle | BGL_8292 | C C | C C | C C | T T |
| Beagle | BGL_8294 | C C | C C | C C | T T |

Table 4.13

| Breed | Sample ID | TLR8_R298Q_chrX_ | TLR8_G442S_chrX_ | TLR8_R686H_chrX_ | TLR9_V87I_chr20_ |
|---|---|---|---|---|---|
| Beagle | BGL_8218 | G G | A A | G A | G G |
| Beagle | BGL_8220-conc | G G | A A | G A | G G |
| Beagle | BGL_8222 | G G | A A | G A | G G |
| Beagle | BGL_8224 | G G | A A | G A | G G |
| Beagle | BGL_8226 | G G | A A | G A | G G |
| Beagle | BGL_8272 | G G | A A | G G | G G |
| Beagle | BGL_8276 | G G | A A | G G | G G |
| Beagle | BGL_8278 | G G | G A | G A | G G |
| Beagle | BGL_8280-50 | G G | G A | G A | G G |
| Beagle | BGL_8284 | G G | G A | G A | G G |
| Beagle | BGL_8286 | G G | G A | G A | G G |
| Beagle | BGL_8288 | G G | A A | A A | G G |
| Beagle | BGL_8290-50 | G G | A A | A A | G G |
| Beagle | BGL_8292 | G G | A A | A A | G G |
| Beagle | BGL_8294 | G G | A A | G A | G G |

Table 4.14

| Breed | Sample ID | TLR9_K381E_chr20 | TLR9_P459T_chr20 | TLR9_S721G_chr20 | TLR9_P787L_chr20 |
|---|---|---|---|---|---|
| Beagle | BGL_8218 | A G | C A | A G | C C |
| Beagle | BGL_8220-conc | G G | A A | G G | C C |
| Beagle | BGL_8222 | G G | A A | A G | C C |
| Beagle | BGL_8224 | G G | A A | A G | C C |
| Beagle | BGL_8226 | G G | A A | G G | C C |
| Beagle | BGL_8272 | G G | A A | A A | C C |
| Beagle | BGL_8276 | G G | A A | G G | C C |
| Beagle | BGL_8278 | G G | A A | A G | C C |
| Beagle | BGL_8280-50 | G G | C A | A G | C C |
| Beagle | BGL_8284 | G G | A A | A G | C C |
| Beagle | BGL_8286 | G G | A A | A G | C C |
| Beagle | BGL_8288 | G G | A A | A G | C C |
| Beagle | BGL_8290-50 | G G | A A | A G | C C |
| Beagle | BGL_8292 | G G | A A | G G | C C |
| Beagle | BGL_8294 | G G | C A | A G | C C |

Table 4.15

| Breed | Sample ID | TLR9_R862W_chr20 | TLR9_328_FRAMESH | TLR10_T361M_chr3 | TLR10_M592V_chr3 |
|---|---|---|---|---|---|
| Beagle | BGL_8218 | C C | * * | C C | A A |
| Beagle | BGL_8220-conc | C C | * * | C C | A A |
| Beagle | BGL_8222 | C C | * * | C C | A A |
| Beagle | BGL_8224 | C C | * * | C C | A A |
| Beagle | BGL_8226 | C C | * * | C C | A A |
| Beagle | BGL_8272 | C C | * * | C C | A A |
| Beagle | BGL_8276 | C C | * * | C C | A A |
| Beagle | BGL_8278 | C C | * * | C C | A A |
| Beagle | BGL_8280-50 | C T | * * | C C | A A |
| Beagle | BGL_8284 | C C | * * | C C | A A |
| Beagle | BGL_8286 | C C | * * | C C | A A |
| Beagle | BGL_8288 | C C | * * | C C | A A |
| Beagle | BGL_8290-50 | C C | * * | C C | A A |
| Beagle | BGL_8292 | C C | * * | C C | A A |
| Beagle | BGL_8294 | C T | * * | C C | A A |

Thus, a simplified identification mode with proper resolution is derivable from the analysis of only some tlr genes, avoiding expensive and more complex methods based on the determination of the whole genome.

The data derivable from the array may be obtained from any sample of a canine subject meanwhile the sample contains DNA or a nucleic acid transformable to DNA (i.e. RNA). Examples of samples are waste material of the subject (defecation and urine), as well as blood, semen, saliva, a biopsy, etc.

A direct use of the information is the identification of samples, in particular of waste material that can be found in the boundaries of streets or in particular gardens. Correct identification (in some cities compulsory) of dogs and their tutors allows further determining the origin of a particular waste material. Identification of the waste material will serve for sanctioning non civic behaviours.

Concluding, with the array and methods of the invention, profile of innate immunity is obtained for each of the analyzed samples. This profile may serve to decide a particular medical regimen for an individual, as well as to conclude a diagnosis. In particular, the suffering from diseases related with dysfunction of innate immunity, due to critical mutations in canine TLR2, TLR5, TLR6 and TLR8.

In addition, an innate immunity profile is a usable tool for the selection of a homogenized population among a canine breed.

The fast and economical detection of the sequences of TLRs by means of the proposed array is advantageous and aims saving cost in research studies or in clinical trials. Indeed, A the TaqMan OpenArray designed for high-throughput genotyping of TLRs in dogs, serves either to study TLR evolution in the canine genome, the association to infection susceptibility or their relationship with either the commensal or the disease associated microbiota.

Further, when the array comprises at least the probes (oligonucleotides) for detecting all mutations listed in Table 1, an individual imprinting of the individual is obtained with enough information to distinct among two individuals. Costs are avoided in relation with a complete genome analysis, and easy manipulation of data is provided with the array and methods of the invention.

### REFERENCES CITED IN THE APPLICATION

Kathrani et al., in “Polymorphisms in the TLr4 and Tlr5 Gene Are Significantly Associated with Inflammatory Bowel Disease in German Shepherd Dogs”, PLoS ONE—2010, Vol. No. 5 (12), pp.: 1-10.
Kathrani et al., “Breed-independent toll-like receptor 5 polymorphisms show association with canine inflammatory bowel disease”, Tissue Antigens—2011, Vol. No. 78, pp.: 94-101
Kumar P et al., “Predicting the effects of coding non-synonymous variants on protein function using the SIFT algorithm”, Nat Protoc.—2009; Vol. No.4(7), pp.:1073-81
Choi Yet al., “Predicting the Functional Effect of Amino Acid Substitutions and Indels”, PLoS ONE—2012, Vol. No. 7(10): e46688.
Adzhubei I a, Schmidt S, Peshkin L, Ramensky V E, Gerasimova A, Bork P, Kondrashov A S, Sunyaev S R: A method and server for predicting damaging missense mutations. Nat Methods 2010, 7:248-9.
Letunic I, Doerks T, Bork P: SMART 7: recent updates to the protein domain annotation resource. Nucleic Acids Res 2012, 40(Database issue):D302-5
Kannaki et al., “Toll-like receptors and their role in animal reproduction”, Animal Reproducing Science—2011, Vol. No. 125 (1-4), pp.:1-12).
Ke et al., “Assessment of the functionality of genome-wide canine SNP arrays and implications for canine disease association studies”, Animal Genetics—2010, Vol. No. 42(2), pp.:181-190.

1. A DNA array comprising:
(i) a canine toll-like receptor 2 (tlr 2) gene fragment that in the entire nucleotide sequence of the gene codifies for a change of a serine (S) by a leucine (L) at position 516 of canine TLR2 polypeptide of SEQ ID NO: 1;
(ii) a canine toll-like receptor 5 (tlr 5) gene fragment that in the entire nucleotide sequence of the gene codifies for a change of a phenylalanine (F) by a cysteine (C) at position 888 of canine TLR5 polypeptide of SEQ ID NO: 2;
(iii) a canine toll-like receptor (tlr 6) gene fragment that in the entire nucleotide sequence of the gene codifies for a change of a leucine (L) by a phenylalanine (F) at position 457 of canine TLR6 polypeptide of SEQ ID NO: 3; and
(iv) a canine toll-like receptor 8 (tlr 8) gene fragment that in the entire nucleotide sequence of the gene codifes for a change of a valine (V) by an alanine (A) at position 157 of canine TLR8 polypeptide of SEQ ID NO: 4.

2. The DNA array of claim 1, wherein the canine tlr gene fragments of (i) to (iv) comprise, respectively, the nucleotide sequences SEQ ID NO: 11 to 13, wherein SEQ ID NO: 11 in the entire nucleotide sequence of tlr 2 gene codifies for the mutation S516L in SEQ ID NO:1; SEQ ID NO: 12 in the entire nucleotide sequence of tlr 6 gene codifies for the mutation L457 in SEQ ID NO: 3; SEQ ID NO: 13 in the entire nucleotide sequence of tlr 8 gene codifies for the mutation V157A in SEQ ID NO: 4; and SEQ ID NO: 47 in the entire nucleotide sequence of tlr 5 gene codifies for the mutation F888C in SEQ ID NO: 2.

3. The DNA array of claim 1, comprising one or more canine tlr gene fragment selected from the group consisting of:
(v) a tlr1 gene fragment that in the entire nucleotide sequence of the gene codifies for a change of a serine (S) by an alanine (A) at position 281, and/or for a change of a valine (V) by an isoleucine (I) at position 312 of TLR1 polypeptide of SEQ ID NO: 5;
(vi) a tlr 2 gene fragment that in the entire nucleotide sequence of the gene codifies for a change of a threonine (T) by a methionine (M) at position 606 of canine TLR2 polypeptide of SEQ ID NO: 1
(vii) a trl 3 gene fragment that in the entire nucleotide sequence of the gene codifies for a change of a glutamic (E) by a glutamic (D) at position 176 of canine TLR3 polypeptide of SEQ ID NO: 6;
(viii) a tlr 4 gene fragment that in the entire nucleotide sequence of the gene codifies for a change of a valine (V) by a methionine (M) at position 82, and/or a change of an alanine (A) by a threonine (T) at position 347, and/or a change of a threonine (T) by an alanine (A) at position 577 of canine TLR4 polypeptide of SEQ ID NO: 7;
(ix) a tlr 5 gene fragment that in the entire nucleotide sequence of the gene codifies for a change of a change of a glutamic (E) by an aspartic (D) at position 169, and/or a change of a serine (S) by an arginine (N) at position 177, and/or a change of a valine (V) by a leucine (L) at position 296, and/or a change of a leucine (L) by a serine (S) at position 383, and/or a change of an arginine (R) by a glutamine (Q) at position 402, and/or a change of an arginine (R) by a glutamine (Q) at position 416, and/or a change of a valine (V) by an isoleucine (I) at position 296, and/or a change of a glycine (G) by a serine (S) at position 533, and/or a change of an arginine (R) by a glutamine (Q) at position 734, and/or a change of a glutamic (D) by a tyrosine (Y) 767, and/or a change of a asparagine by a lysine (K) at position 833, and/or a change of an arginine (R) by a cysteine (C) at position 844, and/or the presence of a leucine (L) instead of a serine (S) at position 850, and/or a change of an alanine by a threonine (T) at position 896, and/or a change of an hystidine (H) by a tyrosine (Y) at position 1017, and/or a change of a glycine (G) by a serine (S) at position 1020, and/or a change of an arginine (R) by a glutamine (Q) at position 1049, and/or a change of an alanine (A) by a threonine (T) at position 1078 of canine TLR5 polypeptide of SEQ ID NO: 2);
(x) a tlr 6 gene fragment that in the entire nucleotide sequence of the gene codifies for a change of a tyrosine (Y) by a cysteine (C) at position 182 of canine TLR6 polypeptide of SEQ ID NO: 3;
(xi) a tlr 10 gene fragment that in the entire nucleotide sequence of the gene codifies for a change of a methionine (M) by a valine (V) at position 592 of canine TLR10 polypeptide of SEQ ID NO: 8;
(xii) a tlr 7 gene fragment that in the entire nucleotide sequence of the gene codifies for a change of an alanine by a glycine at position 16, and/or a change of a phenylalanine (F) by a leucine (L) at position 167, and/or a change of a proline (P) by a leucine (L) at position 1066 of canine TLR7 polypeptide of SEQ ID NO: 9;
(xiii) a tlr 8 gene fragment that in the entire nucleotide sequence of the gene codifies for a change of an arginine (R) by glutamine (Q) at position 298 of canine TLR8 polypeptide of SEQ ID NO: 4;
(xiv) a tlr 9 gene fragment that in the entire nucleotide sequence of the gene codifies for a change of a valine (V) by an isoleucine (I) at position 87, and/or a change of a lysine (K) by a glutamic (E) at position 381, and/or a change of a proline (P) by a threonine (T) at position 459, and/or a proline (P) by a leucine (L) at position 787, and/or an arginine (R) by a tryptophan (W) at position 862 of canine TLR 9 polypeptide of SEQ ID NO: 10, and combinations of said gene fragments.

4. The DNA array of claim 3, wherein the tlr 1 gene fragment of (v) is selected from a group consisting of a sequence comprising SEQ ID NO: 14, a sequence comprising SEQ ID NO: 15, and combinations thereof; the tlr 2 gene fragment of (vi) is a sequence comprising SEQ ID NO: 16; the tlr 3 gene fragment of (vii) is a sequence comprising SEQ ID NO: 17; the tlr 4 gene fragment of (viii) is selected from a group consisting of a sequence comprising SEQ ID NO: 18, a sequence comprising SEQ ID NO: 19, a sequence comprising SEQ ID NO: 20, and combinations thereof; the tlr 5 gene fragment of (ix) is selected from a group consisting of a sequence comprising any of sequences SEQ ID NO: 21-35 and SEQ ID NO: 52, and combinations thereof; the tlr 6 gene fragment of (x) is a sequence comprising SEQ ID NO: 36; the tlr 10 gene fragment of (xi) is a sequence comprising SEQ ID NO: 46; the tlr 7 gene fragment of (xii) is selected from a group consisting of a sequence comprising any of sequences SEQ ID NO: 37-39, and combinations thereof; the tlr 8 gene fragment of (xiii) is a sequence comprising SEQ ID NO: 40; and the tlr 9 gene fragment of (xiv) is selected from a group consisting of a sequence comprising any of sequences 41-45, and combinations thereof.

5. The DNA array of claim 1, comprising one or more canine tlr gene fragments, each gene fragment comprising one or more single nucleotide polymorphisms selected from the group consisting of rs23585044, rs23572381, rs23572380, rs22410121, rs8958543, rs22120766, rs22157966, rs22145736, rs22189454, rs22189456, rs22124023, rs22123995, rs24029590, rs9070448, rs9070450, rs9070451, rs9070452, rs9070447, rs9125247, rs24029975, rs23570247, rs24607342, rs24607358, rs9188882, rs22882109, and rs23518574.

6. The DNA array of claim 1, wherein the canine tlr gene fragments have a length comprised from 10 to 30 nucleotides.

7. The DNA array of claim 1, further comprising primers for amplifying tlr gene fragments selected and included in the array.

8. A method for identifying mutations in canine toll-like receptor genes, the method comprising:
adding an isolated animal sample comprising DNA or cDNA to the DNA array of claim 1; and
determining if the canine toll-like receptor gene fragments hybridize with sample gene fragments.

9. The method of claim 8, wherein the isolated animal sample is selected from the group consisting of blood, saliva, feces, urine, hair, a skin tissue biopsy, and a muscle tissue biopsy.

10. An in vitro method for analyzing a genetic predisposition of an individual of the genus Canis to suffer from a disease related to dysfunction of the innate immunity system, the method comprising:
(a) adding an isolated animal sample comprising DNA or cDNA to the DNA array of claim 1;
(b) determining if the canine toll-like receptor gene fragments present thereon hybridize with the DNA or cDNA; and
(c) correlating any detected mutation with the disease related to dysfunction of the innate immunity system.

11. The in vitro method of claim 10, wherein the disease related to dysfunction of the innate immunity system is selected from the group consisting of chronic enteropathies, osteoarthritis, endometrium infections, sino-nasal aspergillosis, idiopathic lymphoplasmacytic rhinitis, Leishmania infections atopic dermatitis, Inflammatory bowel syndrome, Inflammatory bowel disease, Crohn's disease, viral infections, and bacterial infections.

12. An in vitro method for analyzing a canine innate immunity profile, the method comprising determining the presence or absence of canine toll-like receptor mutations in the canine by contacting a sample comprising DNA from the canine or cDNA derived therefrom with the DNA array of claim 1 and identifying hybridization of the DNA or cDNA to the DNA array.

13. An in vitro method for individually identifying an animal of the genus Canis, the method comprising determining the presence or absence of canine toll-like receptor mutations in an isolated sample using the DNA array of claim 1; and establishing a combination of detected and non-detected mutations in said sample.

14. An in vitro method for distinguishing among individuals of the genus Canis, the method comprising determining the presence or absence of canine toll-like receptor mutations in an isolated sample using the DNA array of claim 3; and establishing a combination of detected and non-detected mutations in said sample.

15. The in vitro method according to of claim 13, wherein the isolated animal sample is selected from the group consisting of blood, saliva, feces, urine, hair, a skin tissue biopsy, and a muscle tissue biopsy.

16. An in vitro method for analyzing variability in canine tlr genes, the method comprising determining the presence or absence of canine toll-like receptor mutations using the DNA array of claim 1.

17. The in vitro method of claim 14, wherein the isolated animal sample is selected from the group consisting of blood, saliva, feces, urine, hair, a skin tissue biopsy, and a muscle tissue biopsy.
